(19) United States
(12) Patent Application Publication
(10) Pub. No.: US 2024/0052331 A1
(43) Pub. Date: Feb. 15, 2024

(54) EVOLUTION OF BOTULINUM NEUROTOXIN PROTEASES

(71) Applicants: The Board Institute, Inc., Cambridge, MA (US); President and Fellows of Harvard College, Cambridge, MA (US); Children's Medical Center Corporation, Boston, MA (US)

(72) Inventors: David R. Liu, Cambridge, MA (US); Travis R. Blum, Cambridge, MA (US); Min Dong, Boston, MA (US); Hao Liu, Boston, MA (US)

(73) Assignees: The Board Institute, Inc., Cambridge, MA (US); President and Fellows of Harvard College, Cambridge, MA (US); Children's Medical Center Corporation, Boston, MA (US)

(21) Appl. No.: 18/268,178

(22) PCT Filed: Dec. 17, 2021
(86) PCT No.: PCT/US21/64125

§ 371 Date: Jun. 16, 2023

Related U.S. Application Data
(60) Provisional application No. 63/127,340, filed on Dec. 18, 2020.

(51) Int. Cl. C12N 9/52; C12N 9/16; C07K 14/47; C12N 15/86

(52) U.S. Cl. CPC C12N 9/52; C12N 9/16; C07K 14/4703; C12N 15/86; C12Y 304/24069; C07K 2319/00; C12Y 301/04011; C12N 2740/15043

(57) ABSTRACT

The disclosure provides fusion proteins comprising a pleckstrin homology (PH) domain and a variant of Botulinum neurotoxin E (BoNT E) protease that cleaves certain non-canonical protein targets (e.g., PTEN). Fusion proteins described in the disclosure are useful for cleaving target proteins found in a cell, that is, in an intracellular environment. Aspects of the disclosure provide methods for inhibiting PTEN amount, activity, or function in a cell or subject, the methods comprising administering to a call or subject a fusion protein described herein.

### CROSS-REFERENCE TO RELATED APPLICATIONS

This application claims the benefit under 35 U.S.C. § 119(e) of the filing date of U.S. Provisional Application Ser. No. 63/127,340, entitled “EVOLUTION OF BOTULINUM NEUROTOXIN PROTEASES”, filed on Dec. 18, 2020; the entire contents of which are incorporated herein by reference.

### FEDERALLY SPONSORED RESEARCH

This invention was made with government support under EB027793, EB022376, GM118062, GM122261, NS080833, and NS106159 awarded by the National Institutes of Health. The government has certain rights in the invention.

### BACKGROUND

Over the last few decades, the medical community has witnessed a remarkable shift in the composition of pharmaceutical therapies from traditional small molecules to biomacromolecules (e.g., enzymes, compositions of multiple proteins, peptides, amino acids, polymers, nucleic acids). The growing number of macromolecular therapeutics is a result of their potential for highly specific interactions in biological systems and has been facilitated by improvements in molecular biology and biomolecule engineering. Despite their tremendous success, macromolecular therapies have been limited almost exclusively to extracellular targets due to the significant challenge of their controllable delivery into the cytoplasm. While a number of notable advances have been made in the area of macromolecular delivery, this important problem remains a major barrier to the development and use of macromolecular therapeutics that address intracellular targets. As an alternative, several natural protein systems are capable of cytoplasmic self-delivery. However, the ability to reengineer these systems to imbue them with the necessary binding or catalytic activities and specificities for therapeutic effect is largely underexplored and underdeveloped.

### SUMMARY

The disclosure relates to fusion proteins comprising certain subcellular localization peptides (e.g., pleckstrin homology (PH) domains, etc.) connected to novel Botulinum neurotoxin (BoNT) protease variants. In some embodiments, the BoNT protease variants have been evolved using Phage-Assisted Continuous Evolution (PACE), for example, as described in U.S. Pat. No. 9,023,594, issued May 5, 2015; U.S. Pat. No. 9,771,574, issued Sep. 26, 2017; U.S. patent application Ser. No. 15/713,403, filed Sep. 22, 2017 (now abandoned); International PCT Application PCT/US2009/056194, filed Sep. 8, 2009, published as WO 2010/028347 on Mar. 11, 2010; U.S. Provisional Patent Application Ser. No. 61/426,139, filed Dec. 22, 2010; U.S. Pat. No. 9,394,537, issued Jul. 19, 2016; U.S. Pat. No. 10,336,997, issued Jul. 2, 2019; U.S. patent application Ser. No. 16/410,767, filed May 13, 2019; International PCT Application PCT/US2011/066747, filed Dec. 22, 2011, published as WO 2012/088381 on Jun. 28, 2012; U.S. Provisional Patent Application Ser. No. 61/929,378 filed Jan. 20, 2014; U.S. Pat. No. 10,179,911, issued Jan. 15, 2019; U.S. patent application Ser. No. 16/238,386, filed Jan. 2, 2019; International PCT Application PCT/US2015/012022, filed Jan. 20, 2015; U.S. Provisional Patent Application Ser. No. 62/158,982, filed May 8, 2015; U.S. Provisional Patent Application Ser. No. 62/187,669, filed Jul. 1, 2015; U.S. Provisional Patent Application Ser. No. 62/067,194, filed Oct. 22, 2014; U.S. Pat. No. 10,920,208, issued Feb. 16, 2021; International PCT Application PCT/US2018/048134, filed Aug. 27, 2018; U.S. Pat. No. 9,267,127, issued Feb. 23, 2016; International PCT Application PCT Application, PCT/US2015/057012, filed Oct. 22, 2015, published as WO 2016/077052; International PCT Application PCT/US2016/027795, filed Apr. 15, 2016, published as WO 2016/168631; International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Provisional Patent Application Ser. No. 62/067,194, filed Oct. 22, 2014, U.S. Pat. No. 9,023,594, issued May 5, 2015, and International PCT Application, PCT/US2018/051557, published as WO 2018/056002 on Mar. 21, 2019, the entire contents of each of which are incorporated herein by reference. As described herein, fusion proteins comprising a PH domain and a BoNT protease light chain variant are attractive candidates for cytosolic delivery of the BoNT protease variant because it has been surprisingly discovered that addition of a PH domain allows the BoNTs to efficiently cleave intracellular targets (e.g., intracellular targets of cells having an intact cell membrane). In some embodiments, the PH domain of the fusion protein directs the BoNT protease to a particular subcellular location (e.g., the plasma membrane) of a cell in order to increase contact of the protease with its target substrate (e.g., a Phosphatase and tensin homolog (PTEN) protein). In some embodiments, the disclosure relates to fusion proteins comprising a PH domain and an evolved BoNT/E protease variant that cleaves a desired substrate (e.g., a disease-associated intracellular protein, such as PTEN protein) are described herein.

Accordingly, in some aspects, the disclosure provides a fusion protein comprising a pleckstrin homology (PH) domain (e.g., SEQ ID NO.: 2, 18, 19, 20, or 21); and a BoNT/E protease light chain having at least 80% (e.g., at least 80%, 85%, 90%, 95%, 99%, etc.) sequence identity to SEQ ID NO.: 1.

In some embodiments, the PH domain is a human PH domain. In some embodiments, a PH domain comprises a human phospholipase C delta 1 (PLCδ1) PH domain. In some embodiments, a PH domain has an amino acid sequence that is at least 80% (e.g., at least 80%, 85%, 90%, 95%, 99%, etc.) identical to the sequence set forth in SEQ ID NO.: 2.

In some embodiments, a BoNT/E protease light chain comprises an amino acid substitution in at least one of the following positions relative to SEQ ID NO. 1: C26, Q27, E28, I35, G49, H56, H56, S99, G101, N118, D156, E159, N161, S162, S163, S166, L167, M172, I203, I232, T242, R244, N248, I262, I263, A313, I316, G353, Q354, Y355, Y357, K359, N365, S367, N390, G403, or L404.

In some embodiments, a BoNT/E protease light chain comprises at least one of the following amino acid substitutions relative to SEQ ID NO.: 1: C26Y, Q27H, E28K, I35V, G49S, H56L, H56Y, S99A, S99T, G101S, N118D, D156N, E159L, N161Y, S162Q, S163R, S166R, M172K, I203V, I232T, T242A, R244V, N248K, I262T, I263V, A313V, I316T, G353E, Q354R, Q354W, Y355P, Y355H, Y357F, K359R, N365S, S367F, N390D, G403E, or L404* (e.g., “L404Stop”).

In some embodiments, a BoNT/E protease comprises the following amino acid substitutions relative to SEQ ID NO.: 1: C26Y, Q27H, S99A, G101S, N118D, D156N, E159L, N161Y, S162Q, S163R, L167A, M172K, I232T, N248K, Q354R, Y355P, and Y357F.

In some embodiments, a fusion protein has at least 80% sequence identity (e.g., at least 80%, at least 85%, at least 90%, at least 95%, at least 96%, at least 97%, at least 98%, at least 99%, at least 99.5%, at least 99.9%, or more) to SEQ ID NO.: 5 or 6.

In some embodiments, a fusion protein comprises or consists of the amino acid sequence set forth in SEQ ID NO.: 5 or 6.

In some embodiments, a PH domain is positioned N-terminal relative to the BoNT/E protease light chain. In some embodiments, a PH domain and a BoNT/E protease light chain are directly connected.

In some embodiments, the fusion protein further comprises a linker, for example, a linker connecting the PH domain to the BoNT/E protease light chain. In some embodiments, the linker comprises a peptide linker. In some embodiments, the peptide linker comprises a glycine-rich linker, a proline-rich linker, glycine/serine-rich linker, or alanine/glutamic acid-rich linker.

In some embodiments, a BoNT/E protease light chain is catalytically active. In some embodiments, a BoNT/E protease light chain is capable of cleaving a non-canonical BoNT/E substrate. In some embodiments, a non-canonical BoNT/E substrate is a Phosphatase and tensin homolog (PTEN) protein (e.g., a protein having an amino acid sequence that is at least 70% identical to the amino acid sequence set forth in SEQ ID NO.: 12 or 13).

In some embodiments, a BoNT/E protease light chain does not cleave a SNAP protein. In some embodiments, a BoNT/E protease light chain does not cleave SNAP25 (e.g., a protein having an amino acid sequence that is at least 70%, 80%, 85%, 90%, 95%, or 99% identical to the amino acid sequence set forth in SEQ ID NO.: 16 or 17).

In some aspects, the disclosure provides an isolated nucleic acid encoding a fusion protein as described herein. In some embodiments, the isolated nucleic acid has at least 60%, 70%, 80%, 90%, 95%, or 99% or more identity to the nucleic acid sequence set forth in SEQ ID NO.: 10 or 11. In some embodiments, an isolated nucleic acid comprises or consists of the nucleic acid sequence set forth in SEQ ID NO.: 10 or 11, which are encoded by SEQ ID NO.: 5 and 6, respectively. In some embodiments, the nucleic acid sequence encoding a fusion protein is codon-optimized. In some embodiments, the nucleic acid sequence is codon-optimized for expression in mammalian (e.g., human) cells.

In some aspects, the disclosure provides a vector comprising an isolated nucleic acid as described herein, for example, an isolated nucleic acid encoding a fusion protein comprising a PH domain and a BoNT/E protease light chain. In some embodiments, a vector is a plasmid or a viral vector. In some embodiments, a viral vector is a lentiviral vector.

In some aspects, the disclosure provides a host cell comprising a fusion protein, isolated nucleic, or vector as described herein. In some embodiments, the cell is a mammalian cell. In some embodiments, the mammalian cell is a human cell. In some embodiments, the cell is in a subject.

In some aspects, the disclosure provides a method of cleaving an intracellular protein, the method comprising delivering to a cell a fusion protein, isolated nucleic acid, or vector as described herein, whereby the fusion protein contacts and cleaves the intracellular protein in the cell. In some embodiments, the intracellular protein is a PTEN protein. In some embodiments, the cell is a mammalian cell. In some embodiments, the cell comprises an intact cell membrane (e.g., the cell has not been permeabilized, the cell is alive, etc.). In some embodiments, the intracellular protein is cleaved in a plasma membrane of a cell.

In some aspects, the disclosure provides a use of a fusion protein, isolated nucleic acid, or vector as described herein in reducing PTEN activity or the amount of functional PTEN in a cell or subject. In some embodiments, the cell is a mammalian cell. In some embodiments, the mammalian cell is a human cell. In some embodiments, the cell is intact. In some embodiments, the cell is in a subject. In some embodiments, the subject is a human. In some embodiments, the cell or subject is characterized as having PTEN activity or expression that is higher than a normal healthy cell or subject

### BRIEF DESCRIPTION OF DRAWINGS

FIGS. 1A-1B show representative data for evaluation of evolved BoNT protease fusion proteins in mammalian cells. FIG. 1A shows evaluation of PTEN cleavage by evolved HA-tagged E(4130)A2 protease after transient co-transfection of plasmids encoding protease and FLAG-tagged PTEN. PH-E(4130)A2 contains an N-terminal pleckstrin homology domain fused to E(4130)A2. The Western blot was visualized using anti-FLAG primary antibodies. Numbers indicate the ratio of BoNT/LC plasmid:PTEN substrate plasmid. FIG. 1B shows a schematic indicating E(4130)A2 amino acid mutations relative to wild-type BoNT/E light chain protease.

FIG. 2 shows representative data for assessment of PTEN and SNAP25 cleavage in HEK293T cells transduced with lentivirus encoding RFP (negative control), PH-E(4130)A2 protease, or the PH-E(4130)A2(L166A) mutant protease; note “L166A” referred to in this Figure corresponds to a mutation at position L167 (e.g., L167A) of SEQ ID NO: 1. After integration, cell lines were transfected with plasmids encoding HA-tagged substrates, and cleavage was visualized by Western blot.

FIG. 3 shows identification of the cleavage site of PTEN by E(4130)A2. Assay was performed using 2 μM MBP-PTEN(N292-N311)-GST substrate, with 100 nM protease, then analyzed by LCMS for average intact mass. PTEN(N292-N311) is indicated in red.

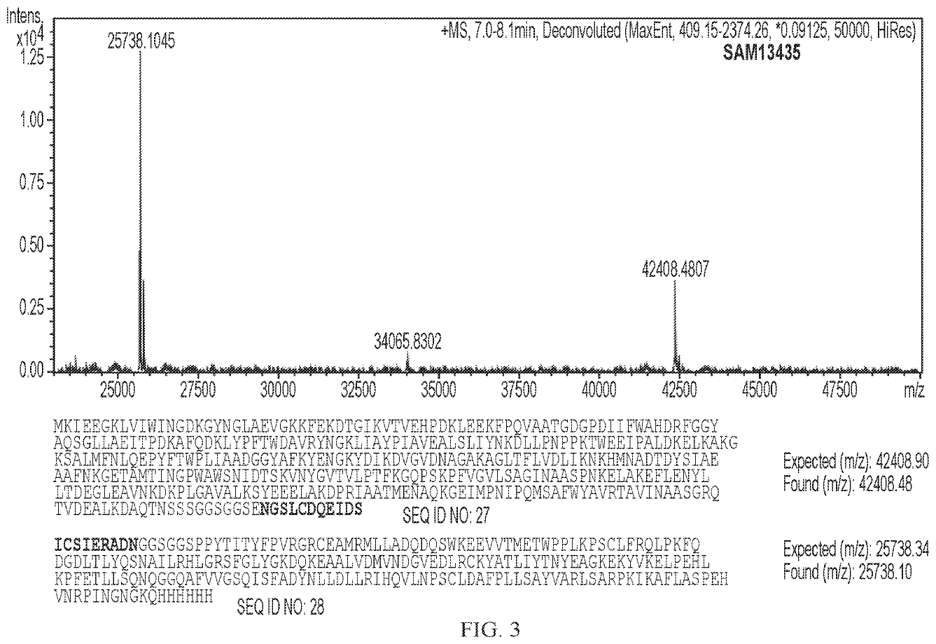

### DEFINITIONS

The term “protein,” as used herein, refers to a polymer of amino acid residues linked together by peptide bonds. The term, as used herein, refers to proteins, polypeptides, and peptides of any size, structure, or function. Typically, a protein will be at least three amino acids long but is generally longer than 50 amino acids in length. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in an inventive protein may be modified, for example, by the addition of a chemical entity such as a carbohydrate group, a hydroxyl group, a phosphate group, a farnesyl group, an isofarnesyl group, a fatty acid group, a linker for conjugation, functionalization, or other modification, etc. A protein may also be a single molecule or may be a multi-molecular complex. A protein may be just a fragment of a naturally occurring protein or peptide. A protein may be naturally occurring, recombinant, or synthetic, or any combination of these.

The term “peptide”, as used herein, refers to a short, contiguous chain of amino acids linked to one another by peptide bonds. Generally, a peptide ranges from about 2 amino acids to about 50 amino acids in length (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length) but may be longer in the case of a polypeptide. In some embodiments, a peptide is a fragment or portion of a larger protein, for example comprising one or more domains of a larger protein. Peptides may be linear (e.g., branched, unbranched, etc.) or cyclic (e.g., form one or more closed rings). A “polypeptide”, as used herein, refers to a longer (e.g., between about 50 and about 100), continuous, unbranched peptide chain.

The term “pleckstrin homology domain” or “PH domain,” as used herein, refers to a polypeptide of roughly 100-120 amino acids in length that binds phosphatidylinositol lipids within biological membranes (e.g., phosphatidylinositol (3,4,5)-trisphosphate and phosphatidylinositol (4,5)-bisphosphate) and proteins, such as the 07-subunits of heterotrimeric G proteins, and protein kinase C. Generally, PH domains function in recruiting and trafficking proteins to different cellular and intracellular membranes. PH domains are found in proteins across several organisms, for example, humans, yeast (e.g., S. cerevisiae) and nematodes (e.g., C. elegans). There are hundreds of proteins that contain PH domains in humans alone. Sequences of PH domains are known in the art, for example as described by European Molecular Biology Lab Protein Family (Pfam) database entry “PF00169” and InterPro database entry IPR001849.

The term “protease,” as used herein, refers to an enzyme that catalyzes the hydrolysis of a peptide (amide) bond linking amino acid residues together within a protein. The term embraces both naturally occurring and engineered proteases. Many proteases are known in the art. Proteases can be classified by their catalytic residue, and protease classes include, without limitation, serine proteases (serine alcohol), threonine proteases (threonine secondary alcohol), cysteine proteases (cysteine thiol), aspartate proteases (aspartate carboxylic acid), glutamic acid proteases (glutamate carboxylic acid), and metalloproteases (metal ion, e.g., zinc). The structures in parentheses correlate to the respective catalytic moiety of the proteases of each class. Some proteases are highly promiscuous and cleave a wide range of protein substrates, e.g., trypsin or pepsin. Other proteases are highly specific and only cleave substrates with a specific sequence. In another example, Botulinum toxin proteases (BoNTs) generally cleave specific SNARE proteins. Proteases that cleave in a very specific manner typically bind to multiple amino acid residues of their substrate. Suitable proteases and protease cleavage sites, also sometimes referred to as “protease substrates,” will be apparent to those of skill in the art and include, without limitation, proteases listed in the MEROPS database, accessible at merops.sanger.ac.uk and described in Rawlings et al., (2014) MEROPS: the database of proteolytic enzymes, their substrates and inhibitors. Nucleic Acids Res 42, D503-D509, the entire contents of each of which are incorporated herein by reference. The disclosure is not limited in this respect.

The term “Botulinum neurotoxin (BoNT) protease,” as used herein, refers to a protease derived from, or having at least 70% sequence homology to (or at least 70% identity to) a Botulinum neurotoxin (BoNT), for example, a BoNT derived from a bacterium of the genus Clostridium (e.g., C. botulinum). Structurally, BoNT proteins comprise two conserved domains, a “heavy chain” (HC) and a “light chain” (LC). The LC comprises a zinc metalloprotease domain responsible for the catalytic activity of the protein. The HC typically comprises an HCC domain, which is responsible for binding to neuronal cells, and an HCN domain, which mediates translocation of the protein into a cell. Examples of BoNT HC domains are represented by the amino acid sequences set forth in SEQ ID NOs.: 14 and 15 below.

BoNT E HCC Domain
SSVLNMRYKNDKYVDTSGYDSNININGDVYKYPTNKNQFEIYNDKLSEV
NISQNDYIIYDNKYKNFSISFWVRIPNYDNKIVNVNNEYTIINCMRDNN
SGWKVSLNHNEIIWTLQDNAGINQKLAFNYGNANGISDYINKWIFVTIT
NDRLGDSKLYINGNLIDQKSILNLGNIHVSDNILFKIVNCSYTRYIGIR
YFNIFDKELDETEIQTLYSNEPNTNILKDFWGNYLLYDKEYYLLNVLKP
NNFIDRRKDSTLSINNIRSTILLANRLYSGIKVKIQRVNNSSTNDNLVR
KNDQVYINFVASKTHLFPLYADTATTNKEKTIKISSSGNRFNQVVVMNS
VGNNCTMNFKNNNGNNIGLLGFKADTVVASTWYYTHMRDHTNSNGCFWN
FISEEHGWQEK (BpNT E HC_{C}, Binding domain; SEQ ID
NO.: 15)
BoNT E HCN Domain
CKNIVSVKGIRKSICIEINNGELFFVASENSYNDDNINTPKEIDDTVTS
NNNYENDLDQVILNFNSESAPGLSDEKLNLTIQNDAYIPKYDSNGTSDI
EQHDVNELNVFFYLDAQKVPEGENNVNLTSSIDTALLEQPKIYTFFSSE
FINNVNKPVQAALFVSWIQQVLVDFTTEANQKSTVDKIADISIVVPYIG
LALNIGNEAQKGNFKDALELLGAGILLEFEPELLIPTILVFTIKSFLGS
SDNKNKVIKAINNALKERDEKWKEVYSFIVSNWMTKINTQFNKRKEQMY
QALQNQVNAIKTIIESKYNSYTLEEKNELTNKYDIKQIENELNQKVSIA
MNNIDRFLTESSISYLMKLINEVKINKLREYDENVKTYLLNYIIQHGSI
LGESQQELNSMVTDTLNNSIPFKLSSYTDDKILISYFNKFFKRIKS
(BoNT E HC_{N}, translocation domain; SEQ ID NO.: 14)

There are seven serotypes of BoNTs, denoted BoNT A-G. BoNT serotypes A, C, and E cleave synaptosome-associated protein (SNAP25). BoNT serotype C has also been observed to cleave syntaxin. BoNT serotypes B, D, F, and G cleave vesicle-associated membrane proteins (VAMPs). An example of a SNAP25 protein that is cleaved by wild-type BoNT proteases (e.g., BoNT E) is represented by the amino acid sequence set forth in SEQ ID NO.: 16 below. In some embodiments, a SNAP25 substrate that is cleaved by wild-type BoNT proteases comprises the following amino acid sequence RQIDRIMEKA (SEQ ID NO: 17).

### SNAP25 Protein Sequence

(SEQ ID NO.: 16)
MAEDADMRNELEEMQRRADQLADESLESTRRMLQLVEESKDAGIRTLVM
LDEQGEQLERIEEGMDQINKDMKEAEKNLTDLGKFCGLCVCPCNKLKSS
DAYKKAWGNNQDGVVASQPARVVDEREQMAISGGFIRRVTNDARENEMD
ENLEQVSGIIGNLRHMALDMGNEIDTQNRQIDRIMEKADSNKTRIDEAN
QRATKMLGSG

A wild-type BoNT protease refers to the amino acid sequence of a BoNT protease as it naturally occurs in a Clostridium botulinum genome. A non-limiting example of a wild-type BoNT/E protease light chain sequence is represented by the amino acid sequence set forth in SEQ ID NO.: 1.

The term “BoNT protease variant,” as used herein, refers to a protein (e.g., a BoNT protease) having one or more amino acid variations introduced into the amino acid sequence, e.g., as a result of application of the PACE method or by genetic engineering (e.g., recombinant gene expression, gene synthesis, etc.), as compared to the amino acid sequence of a naturally-occurring or wild-type BoNT protein (e.g., SEQ ID NO.: 1). Amino acid sequence variations may include one or more mutated residues within the amino acid sequence of the protease, e.g., as a result of a change in the nucleotide sequence encoding the protease that results in a change in the codon at any particular position in the coding sequence, the deletion of one or more amino acids (e.g., a truncated protein), the insertion of one or more amino acids, or any combination of the foregoing. In certain embodiments, a BoNT protease variant cleaves a different target peptide (e.g., has broadened or different substrate specificity) relative to a wild-type BoNT protease. For example, in some embodiments, a BoNT/E protease variant cleaves a PTEN protein or peptide.

The term “continuous evolution,” as used herein, refers to an evolution procedure, in which a population of nucleic acids is subjected to multiple rounds of (a) replication, (b) mutation (or modification of the primary sequence of nucleotides of the nucleic acids in the population), and (c) selection to produce a desired evolved product, for example, a novel nucleic acid encoding a novel protein with a desired activity, wherein the multiple rounds of replication, mutation, and selection can be performed without investigator interaction, and wherein the processes (a)-(c) can be carried out simultaneously. Typically, the evolution procedure is carried out in vitro, for example, using cells in culture as host cells. In general, a continuous evolution process provided herein relies on a system in which a gene of interest is provided in a nucleic acid vector that undergoes a life-cycle including replication in a host cell and transfer to another host cell, wherein a critical component of the life-cycle is deactivated and reactivation of the component is dependent upon a desired variation in an amino acid sequence of a protein encoded by the gene of interest, for example, a gene encoding s BoNT/E protease light chain.

The term “phage-assisted continuous evolution (PACE),” as used herein, refers to continuous evolution that employs phage as viral vectors. PACE methods are known in the art and are described, for example, in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; and U.S. Application, U.S. Ser. No. 13/922,812, filed Jun. 20, 2013, each of which is incorporated herein by reference.

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, 0(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′-N-phosphoramidite linkages).

An “isolated nucleic acid” generally refers to refers to a nucleic acid that is: (i) amplified in vitro by, for example, polymerase chain reaction (PCR); (ii) recombinantly produced by molecular cloning; (iii) purified, as by restriction endonuclease cleavage and gel electrophoretic fractionation, or column chromatography; or (iv) synthesized by, for example, chemical synthesis. An isolated nucleic acid is one which is readily manipulatable by recombinant DNA techniques known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulatable by standard techniques known to those of ordinary skill in the art. As used herein with respect to proteins or peptides, the term “isolated” refers to a protein or peptide that has been isolated from its natural environment or artificially produced (e.g., by chemical synthesis, by recombinant DNA technology, etc.).

The term “vector,” as used herein, refers to any genetic element, such as a plasmid, phage, transposon, cosmid, chromosome, artificial chromosome, virus, virion, etc., which is capable of replication when associated with the proper control elements, and which can transfer gene sequences between cells.

The term “viral vector,” as used herein, refers to a nucleic acid (or isolated nucleic acid) comprising a viral genome that, when introduced into a suitable host cell, can be replicated and packaged into viral particles able to transfer the viral genome into another host cell. The term viral vector extends to vectors comprising truncated or partial viral genomes. For example, in some embodiments, a viral vector is provided that lacks a gene encoding a protein essential for the generation of infectious viral particles or for viral replication. In some embodiments, a viral vector is a lentiviral vector, adenoviral vector, or an adeno-associated virus vector.

The term “host cell,” as used herein, refers to a cell that can host a viral vector useful for a continuous evolution process as provided herein. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the invention is not limited in this respect.

In some embodiments, modified viral vectors are used in continuous evolution processes as provided herein. In some embodiments, such modified viral vectors lack a gene required for the generation of infectious viral particles. In some such embodiments, a suitable host cell is a cell comprising the gene required for the generation of infectious viral particles, for example, under the control of a constitutive or a conditional promoter (e.g., in the form of an accessory plasmid, as described herein). In some embodiments, the viral vector used lacks a plurality of viral genes. In some such embodiments, a suitable host cell is a cell that comprises a helper construct providing the viral genes required for the generation of viral particles. A cell is not required to actually support the life cycle of a viral vector used in the methods provided herein. For example, a cell comprising a gene required for the generation of infectious viral particles under the control of a conditional promoter may not support the life cycle of a viral vector that does not comprise a gene of interest able to activate the promoter, but it is still a suitable host cell for such a viral vector. In some embodiments, the viral vector is a phage, and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, XL1-Blue MRF′, and DH10B. In some embodiments, the strain of E. coli used is known as S1030 (available from Addgene). In some embodiments, the strain of E. coli use to express proteins is BL21(DE3). These strain names are art recognized, and the genotype of these strains has been well characterized. It should be understood that the above strains are exemplary only, and that the invention is not limited in this respect.

The term “promoter” refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active under specific conditions. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

The term “cell,” as used herein, refers to a cell derived from an individual organism, for example, from a mammal. A cell may be a prokaryotic cell or a eukaryotic cell. In some embodiments, the cell is a eukaryotic cell, for example, a human cell, a mouse cell, a pig cell, a hamster cell, a monkey cell, etc. In some embodiments, the cell is obtained from a subject having or suspected of having a disease characterized by increased PTEN levels/activity, for example, ischemic neuronal injury (stroke). In some embodiments, the cell is in a subject (e.g., the cell is in vivo). In some embodiments, the cell is intact (e.g., the outer membrane of the cell, such as the plasma membrane, is intact or not permeabilized).

The term “intracellular environment,” as used herein, refers to the aqueous biological fluid (e.g., cytosol) forming the microenvironment contained by the outer membrane of a cell. For example, in a subject, an intracellular environment may include the cytoplasm of a cell or cells of a target organ or tissue (e.g., the cytosol of neuronal cells in CNS tissue). In another example, a cellular environment is the cytoplasm of a cell or cells surrounded by cell culture growth media housed in an in vitro culture vessel, such as a cell culture plate or flask.

The term “subject,” as used herein, refers to an individual organism, for example, a mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cow, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject has a disease characterized by increased activity of an intracellular protein (e.g., a SNARE protein, PTEN, etc.).

The “percent identity” of two amino acid sequences may be determined using algorithms or computer programs, for example, the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into various computer programs, for example NBLAST and XBLAST programs (version 2.0) of Altschul et al. J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul, S F et al., (1997) Nuc. Acids Res. 25: 3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

### DETAILED DESCRIPTION

Aspects of the disclosure relate to compositions and methods for cleaving intracellular protein targets. The disclosure is based, in part, on the surprising discovery that appending a pleckstrin homology (PH) domain to a BoNT/E protease light chain variant results in a fusion protein that 1) localizes the protease variant to the correct subcellular location, and 2) cleaves the protein target of the variant at that subcellular location. In some embodiments, the BoNT/E protease light chain variant has been evolved (e.g., using PACE) to cleave a non-canonical BoNT/E substrate, for example, a PTEN protein. In some embodiments, the evolved BoNT/E protease light chain variant has activity toward a non-canonical substrate (e.g., a PTEN protein) while simultaneously losing its activity to its native substrate (a SNAP25 protein). In some embodiments, fusion proteins described by the disclosure are useful for cleaving certain protein targets (e.g., PTEN) localized to a particular intracellular compartment, for example, a cell's plasma membrane.

### Pleckstrin Homology (PH) Domains

In some aspects, the disclosure relates to fusion proteins comprising a pleckstrin homology (PH) domain. In some embodiments, a PH domain mediates binding to a biological membrane, for example, a plasma membrane of a cell. In some embodiments, a PH domain binds to phosphatidylinositol lipids within the biological membrane and/or certain proteins, such as the fr-subunits of heterotrimeric G proteins or protein kinase C. Without wishing to be bound by any particular theory, inclusion of one or more PH domains in a fusion protein enables the fusion protein to be localized to certain subcellular locations, for example, the plasma membrane of a cell.

In some embodiments, a PH domain is derived from a eukaryotic protein. In some embodiments, a PH domain comprises an amino acid sequence that is at least 80% identical to the sequence set forth in SEQ ID NO.: 2. Additional examples of PH domains include, but are not limited to, the human cytohesin-1 PH domain, human cytohesin-2 PH domain, human cytohesin-3 PH domain, and tyrosine-protein kinase BTK PH domain. Examples of PH domain amino acid sequences are set forth in SEQ ID NOs.: 18-21. In some embodiments, a PH domain comprises an amino acid sequence that is at least 80% identical to the sequence set forth in SEQ ID NOs: 18-21.

The amount or level of variation between two PH domains provided herein can be expressed as the percent identity of the nucleic acid sequences or amino acid sequences between the two nucleic acids or proteins. In some embodiments, the amount of variation is expressed as the percent identity at the amino acid sequence level. In some embodiments, the percent identity is calculated based upon a comparison of the PH domain sequence with a reference PH domain sequence (e.g., SEQ ID NO.: 2).

In some embodiments, a PH domain used in the fusion proteins described herein and the reference PH domain are from about 70% to about 99.9% identical, about 75% to about 95% identical, about 80% to about 90% identical, about 85% to about 95% identical, or about 95% to about 99% identical at the amino acid sequence level. In some embodiments, a PH domain used in the fusion proteins described herein comprises an amino acid sequence that is at least 85%, at least 90%, at least 95%, at least 99%, or at least 99.9% identical to the amino acid sequence of the PH domain represented by the amino acid sequence set forth in SEQ ID NO: 2.

In some embodiments, a PH domain used in the fusion proteins described herein comprises an amino acid sequence that is at least 85%, at least 90%, at least 95%, at least 99%, or at least 99.9% identical to the amino acid sequence of the PH domain represented by the amino acid sequence set forth in SEQ ID NO: 18.

In some embodiments, a PH domain used in the fusion proteins described herein comprises an amino acid sequence that is at least 85%, at least 90%, at least 95%, at least 99%, or at least 99.9% identical to the amino acid sequence of the PH domain represented by the amino acid sequence set forth in SEQ ID NO: 19.

In some embodiments, a PH domain used in the fusion proteins described herein comprises an amino acid sequence that is at least 85%, at least 90%, at least 95%, at least 99%, or at least 99.9% identical to the amino acid sequence of the PH domain represented by the amino acid sequence set forth in SEQ ID NO: 20.

In some embodiments, a PH domain used in the fusion proteins described herein comprises an amino acid sequence that is at least 85%, at least 90%, at least 95%, at least 99%, or at least 99.9% identical to the amino acid sequence of the PH domain represented by the amino acid sequence set forth in SEQ ID NO: 21.

Some aspects of the disclosure provide PH domains having between 1 and 20 amino acid differences (e.g., mutations, substitutions, deletions, insertions, etc.) relative to a reference PH domain (e.g., SEQ ID NO.: 2, 18, 19, 20, or 21). In some embodiments, a PH domain has 1, 2, 3, 4, 5, 6, 7, 8, 9, 10, 11, 12, 13, 14, or 15 amino acid differences relative to a reference PH domain (e.g., SEQ ID NO.: 2, 18, 19, 20, or 21).

### Botulinum Neurotoxin (BoNT) Protease Variants

This disclosure provides fusion proteins comprising variants of BoNT proteases that are derived from a wild-type BoNT E protease (e.g., SEQ ID NO.: 1). In some embodiments, the BoNT protease has at least one of the amino acid variations present in Table 1 (or comprises one or more mutations at a position corresponding to the amino acid variations present in Table 1). Additional examples of BoNT proteases that can be included in fusion proteins are described in PCT Publication WO 2019/040935, published Feb. 28, 2019 and PCT Publication WO 2021/011579, published Jan. 21, 2021, the entire contents of each of which are incorporated herein by reference. In some embodiments, a BoNT protease variant is a BoNT light chain protease variant (e.g., the variant does not comprise a BoNT heavy chain peptide or polypeptide). The variation in amino acid sequence generally results from a mutation, insertion, or deletion in a DNA coding sequence. Mutation of a DNA sequence can result in a nonsense mutation (e.g., a transcription termination codon (TAA, TAG, or TAA) that produces a truncated protein), a missense mutation (e.g., an insertion or deletion mutation that shifts the reading frame of the coding sequence), or a silent mutation (e.g., a change in the coding sequence that results in a codon that codes for the same amino acid normally present in the cognate protein, also referred to sometimes as a synonymous mutation). In some embodiments, mutation of a DNA sequence results in a non-synonymous (i.e., conservative, semi-conservative, or radical) amino acid substitution.

TABLE 1
Substitution relative to SEQ ID NO.: 1

| | | | | | |
|---|---|---|---|---|---|
| C26Y | S99A/T | S163R | R244V | Q354R/W | G403E |
| Q27H | G101S | S166R | N248K | Y355P | L404* |
| | | | | | (Stop) |
| E28K | N118D | L167A | I262T | Y357F | |
| I35V | D156N | M172K | I263V | K359R | |
| G49S | E159L | I203V | A313V | N365S | |
| H56L/Y | N161Y | I232T | I316T | S367F | |
| D65G | S162Q | T242A | G353E | N390D | |

Generally, wild-type BoNT protease is encoded by a gene of the microorganism Clostridium botulinum. The amount or level of variation between a wild-type BoNT protease and a BoNT protease variant provided herein can be expressed as the percent identity of the nucleic acid sequences or amino acid sequences between the two genes or proteins. In some embodiments, the amount of variation is expressed as the percent identity at the amino acid sequence level. In some embodiments, the percent identity is calculated based upon the sequences of the wild-type and variant protease light chains (e.g., the heavy chain sequences are not aligned or included in the calculation of percent identity).

In some embodiments, a BoNT protease light chain variant and a wild-type BoNT protease light chain are from about 70% to about 99.9% identical, about 75% to about 95% identical, about 80% to about 90% identical, about 85% to about 95% identical, or about 95% to about 99% identical at the amino acid sequence level. In some embodiments, a BoNT protease light chain variant comprises an amino acid sequence that is at least 85%, at least 90%, at least 95%, at least 99%, or at least 99.9% identical to the amino acid sequence of a wild-type BoNT protease light chain.

In some embodiments, a variant BoNT protease is about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 99.9% identical to a wild-type BoNT protease. In some embodiments, a variant BoNT protease is not 100% identical to SEQ ID NO: 1.

Some aspects of the disclosure provide variant BoNT proteases having between about 90% and about 99.9% (e.g., about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.2%, about 99.4%, about 99.6%, about 99.8%, or about 99.9%) identical to a wild-type BoNT protease as set forth in SEQ ID NO.: 1. In some embodiments, the variant BoNT protease is no more than 99.9% identical to a wild-type BoNT protease.

Some aspects of the disclosure provide variant BoNT protease light chains having between 1 and 20 amino acid substitutions (e.g., mutations) relative to a wild-type BoNT protease light chain (e.g., SEQ ID NO.: 1). In some embodiments, a variant BoNT protease has 1, 2, 3, 4, 5, 6, 7, 8, 9, 10, 11, 12, 13, 14, or 15 amino acid substitutions relative to a wild-type BoNT protease (e.g., SEQ ID NO.: 1). In some embodiments, a variant BoNT protease has at least one mutation relative to a wild-type BoNT protease (e.g., SEQ ID NO.: 1).

The amount or level of variation between a wild-type BoNT protease and a variant BoNT protease can also be expressed as the number of mutations present in the amino acid sequence encoding the variant BoNT protease relative to the amino acid sequence encoding the wild-type BoNT protease. In some embodiments, an amino acid sequence encoding a variant BoNT protease comprises between about 1 mutation and about 40 mutations, about 10 mutations and about 20 mutations, about 5 mutations and about 15 mutations, about 2 mutations and about 25 mutations, or about 15 and about 30 mutations relative to an amino acid sequence encoding a wild-type BoNT protease. In some embodiments, an amino acid sequence encoding a variant BoNT protease comprises more than 40 mutations relative to an amino acid sequence encoding a wild-type BoNT protease. In some embodiments, the variant BoNT protease comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 amino acid variations at one or more amino acid positions selected from the positions provided in Table 1. In some embodiments, the variant BoNT protease comprises at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, or 37 amino acid variations selected from the variations (e.g., amino acid substitutions) provided in Table 1.

Particular combinations of mutations present in an amino acid sequence encoding a variant BoNT protease light chain can be referred to as the “genotype” of the variant BoNT protease. For example, a variant BoNT E protease light chain genotype may comprise the mutations C26Y, Q27H, S99A, G101S, N118D, D156N, E159L, N161Y, S162Q, S163R, L167A, M172K, I232T, N248K, Q354R, Y355P, and Y357F, relative to a wild-type BoNT E protease (e.g., SEQ ID NO.: 1; wild-type BoNT E). In some embodiments, a fusion protein has at least 80% sequence identity (e.g., at least 70%, at least 71%, at least 72%, at least 73%, at least 74%, at least 75%, at least 76%, at least 77%, at least 78%, at least 79%, at least 80%, at least 85%, at least 90%, at least 95%, at least 96%, at least 97%, at least 98%, at least 99%, at least 99.5%, at least 99.9%, or more) to SEQ ID NO.: 5 or 6. In some embodiments, a fusion protein comprises or consists of the amino acid sequence set forth in SEQ ID NO.: 5 or 6.

This disclosure relates, in part, to the discovery that continuous evolution methods (e.g., PACE) are useful for producing BoNT protease variants that have altered peptide cleaving activities (altered peptide cleaving functions). For example, in some embodiments, a BoNT protease variant as described by the disclosure cleaves a PTEN protein or peptide. In some embodiments, a PTEN protein or peptide comprises the amino acid sequence set forth in SEQ ID NO.: 12 or 13. In some embodiments, a PTEN protein or peptide comprises an amino acid sequence that is at least 70%, 80%, 85%, 90%, 95%, 99%, or more identical to the amino acid sequence set forth in SEQ ID NO.: 12 or 13.

In some embodiments, a BoNT protease variant cleaves a target peptide (e.g., PTEN, etc.) with higher activity than a wild-type BoNT protease. A BoNT protease variant that cleaves a target peptide (e.g., PTEN, etc.) with higher activity can have an increase in catalytic efficiency ranging from about 1.1-fold, about 1.5-fold, 2-fold to about 100-fold, about 5-fold to about 50-fold, or about 10-fold to about 40-fold, relative to the catalytic efficiency of the wild-type BoNT protease from which the BoNT protease variant was derived. In some embodiments, a BoNT protease variant described herein cleaves a target peptide (e.g., PTEN, etc.) with about 1% to about 100% (e.g., about 1%, 2%, 5%, 10%, 20%, 50%, 80%, 90%, 100%) of the catalytic efficiency with which wild-type BoNT cleaves its native substrate (e.g., SNAP25, VAMP1, etc.). Catalytic efficiency can be measured or determined using any suitable method known in the art, for example, using the methods described in Harris et al. (2009) Methods Enzymol. 463; 57-71.

In some aspects, the disclosure relates to BoNT/E protease light chain variants comprising one or more mutations that affect substate specificity of the protease. It has been observed that position L167 (also referred to as “L166” when the wild-type BoNT/E protease sequence does not comprise an N-terminal methionine) plays an important role in SNAP25 binding and cleavage by the protease. Substituting an alanine at this position has been demonstrated to impair substrate binding and catalysis of SNAP25 by BoNT/E, as described by Chen and Barbieri, J Biol Chem. 2007 Aug. 31; 282(35):25540-7. Without wishing to be bound by any particular theory, inclusion of a L167A mutation in a BoNT/E protease variant light chain described herein reduces “off-target” (e.g., SNAP25) cleavage by proteases variants evolved to cleave another target (e.g., PTEN). In some embodiments, a BoNT/E protease light chain variant comprises a L167A (with respect to SEQ ID NO.: 1) substitution.

Generally, the evolution of proteases with altered specificity has focused exclusively on the destruction of therapeutically relevant extracellular proteins. However, fusion proteins comprising BoNTs described herein provide a built-in cytosolic delivery mechanism, and thus are able, in some embodiments, to degrade intracellular targets. For example, in some embodiments, a fusion protein comprising a BoNT protease variant as described herein comprises one or more protein domains that facilitate transport of the protease across a cellular membrane. In some embodiments, the one or more protein domains that facilitate transport across the membrane comprise a pleckstrin homology (PH) domain. In some embodiments, BoNT protease variants described by the disclosure are capable of crossing the cellular membrane and entering the intracellular environment of neuronal cell types.

### Fusion Proteins

Aspects of the disclosure relate to fusion proteins. In some embodiments, the disclosure provides a fusion protein for use in cleaving an intracellular protein (e.g., PTEN), comprising delivering to a cell the fusion protein described herein, whereby the fusion protein contacts and cleaves the intracellular protein in the cell. A fusion protein generally refers to a protein comprising a first peptide derived from a first protein that is linked in a contiguous chain to a second peptide derived from a second protein that is different than the first protein. The first and second peptides may be linked directly (e.g., the C-terminus of the first peptide may be directly linked, such as by a peptide bond, to the N-terminus of the second peptide, or vice versa) or indirectly (e.g., the first peptide and second peptide are joined by a linking molecule, such as an amino acid or polymeric linker).

In some embodiments, a fusion protein comprises a PH domain linked to a BoNT/E protease light chain variant. In some embodiments, the PH domain and the BoNT/E protease light chain variant are directly linked together (e.g., the two peptides are bonded together without an intervening linker sequence). In some embodiments, the C-terminus of the PH domain is linked to the N-terminus of the BoNT/E protease light chain variant. In some embodiments, the BoNT/E protease light chain variant is modified to lack an N-terminal methionine residue.

In some embodiments, a PH domain is indirectly linked to a BoNT/E protease light chain variant via a linker. A linker is generally a peptide linker, for example, a glycine-rich linker (e.g., a poly-glycine-serine linker) or a proline-rich linker (e.g., a poly-Pro linker). The length of the linker may vary. In some embodiments, a linker ranges from about two amino acids in length to about 50 amino acids in length. In some embodiments, a linker comprises 2, 3, 4, 5, 6, 7, 8, 9, 10, 11, 12, 13, 14, 15, 16, 17, 18, 19, 20, 21, 22, 23, 24, or 25 amino acids. In some embodiments, a linker comprises more than 25 amino acids, for example 30, 35, 40, 45, or 50 amino acids. In some embodiments, a linker is a non-peptide linker, for example a polypropylene linker, polyethylene glycol (PEG) linker, etc.).

A fusion protein may be encoded by an isolated nucleic acid or a vector. In some embodiments, the disclosure provides an isolated nucleic acid for use in cleaving an intracellular protein, comprising delivering to a cell the isolated nucleic acid described herein, whereby the fusion protein contacts and cleaves the intracellular protein in the cell. In some embodiments, an isolated nucleic acid encoding a fusion protein further comprises one or more promoters that control expression of the fusion protein. The one or more promoters may be constitutive promoter(s), inducible promoter(s), tissue-specific promoters, or any combination of the foregoing. In some embodiments, an isolated nucleic acid encoding a fusion protein described herein further comprises a human cytomegalovirus (CMV) promoter that controls expression of the fusion protein. In some embodiments, an isolated nucleic acid encoding a fusion protein described herein further comprises a human synapsin 1 promoter that controls expression of the fusion protein.

In some embodiments, an isolated nucleic acid encoding a fusion protein is comprised in a vector, such as a plasmid or viral vector. In some embodiments, the disclosure provides a vector for use in cleaving an intracellular protein, comprising delivering to a cell the vector described herein, whereby the fusion protein contacts and cleaves the intracellular protein in the cell. In some embodiments the viral vector is a lentiviral vector. “Lentivirus” generally refers a family of retroviruses that cause chronic and severe infections in mammalian species. Lentiviruses infect and integrate their genomes into dividing and non-dividing cells (e.g., neurons). Non-limiting examples of lentiviruses used for vectors include human immunodeficiency virus (HIV), simian immunodeficiency virus (SIV), feline immunodeficiency virus (FIV), equine infectious anemia virus (EIAV), bovine immunodeficiency virus (BIV) and caprine arthritis encephalitis virus (CAEV). In some embodiments, lentiviral TRs are derived from HIV (e.g., share at least 50%, 60%, 70%, 80%, 90%, 95%, 99%, or 100% nucleic acid sequence identity with an HIV TR), for example, as described by Chung et al., Mol Ther. 2014 May; 22(5): 952-963.

In some aspects, provided herein is a kit comprising a container housing the fusion protein provided herein, the isolated nucleic acid provided herein, the vector provided herein, or the host cell provided herein.

### Methods and Uses

Some aspects of this disclosure provide methods for using a fusion protein provided herein. In some embodiments, the methods include contacting a protein comprising a protease target cleavage sequence (e.g., PTEN cleavage sequence, SEQ ID NO: 13), for example, ex vivo, in vitro, or in vivo (e.g., in a subject), with the fusion protein, whereby the protease portion of the fusion protein cleaves the protein target. In some embodiments, the therapeutic target is PTEN. Generally, PTEN is an intracellular protein comprising a tensin domain and a phosphatase domain that functions as a tumor suppressor. PTEN has also been observed to mediate ischemic neuronal damage after a stroke. Accordingly, in some aspects, the disclosure provides methods of decreasing PTEN activity in a cell (e.g., reducing the amount of intact or functional PTEN in a cell), the method comprising contacting the cell with, or introducing into the intracellular environment, a fusion protein as described herein (e.g., a fusion protein comprising a PH domain linked to a BoNT/E variant that cleaves PTEN).

In some embodiments, the cell (or intracellular environment) is characterized by increased, aberrant, or undesired activity of a target protein (e.g., PTEN, etc.) relative to a normal cell. In some embodiments, increased activity of a target protein (e.g., PTEN, etc.) occurs when, in a cell, the activity of the target protein is about 2-fold, 3-fold, 4-fold, 5-fold, 10-fold, 25-fold, 50-fold, 100-fold, 500-fold, or 1000-fold over activity of the target protein in a normal healthy cell. In some embodiments, a cell characterized by increased expression of a target protein (e.g., PTEN, etc.) is derived from a subject (e.g., a mammalian subject, such as a human or mouse) that has or is suspected of having a disease associated with increased activity of the target gene, for example, cancer or neuronal damage in the context of PTEN overexpression or increased activity.

In some embodiments, the methods provided herein comprise contacting (e.g., cleaving) the target protein (e.g., PTEN, etc., or a protein comprising a peptide comprising an amino acid sequence that is at least 70%, 80%, 90%, 95%, 99% or more identical with the amino acid sequence set forth in SEQ ID NO.: 12 or 13) with a fusion protein described herein in vitro. In some embodiments, the methods provided herein comprise contacting the target protein with the protease variant described herein in vivo. In some embodiments, the methods provided herein comprise contacting the target protein (e.g., PTEN, etc., or a protein comprising a peptide comprising an amino acid sequence set forth in SEQ ID NO.: 12 or 13 with a fusion protein described herein in a cell or an intracellular environment. In some embodiments, the methods provided herein comprise contacting the target protein (e.g., PTEN, etc., or a protein comprising a peptide comprising an amino acid sequence set forth in SEQ ID NO.: 12 or 13 with a fusion protein in a subject, e.g., by administering the fusion protein to the subject, either locally or systemically. In some such embodiments, the fusion protein is administered to the subject in an amount effective to result in a measurable decrease in the level of full-length (or functional) target protein (e.g., etc.) in the subject, or in a measurable increase in the level of a cleavage product generated by the protease variant upon cleavage of the target protein. In some embodiments, the decrease in the level of full-length (or functional) target protein (e.g., VAMP7, etc.) is at least 10% or more (e.g., at least 10%, at least 30%, at least 40%, at least 50%, at least 60%, at least 70%, at least 80%, at least 85%, at least 90%, at least 95%, at least 96%, at least 97%, at least 98%, at least 99%, or more). In some embodiments, administration of a fusion protein described herein does not result in cleavage of proteins or peptides other than PTEN.

### Host Cells

Some aspects of this invention relate to host cells for continuous evolution processes as described herein. In some embodiments, a host cell is provided that comprises at least one viral gene encoding a protein required for the generation of infectious viral particles under the control of a conditional promoter, and a fusion protein comprising a transcriptional activator targeting the conditional promoter and fused to an inhibitor via a linker comprising a protease cleavage site. For example, some embodiments provide host cells for phage-assisted continuous evolution processes, wherein the host cell comprises an accessory plasmid comprising a gene required for the generation of infectious phage particles, for example, M13 gIII, under the control of a conditional promoter, as described herein. In some embodiments, the host cells comprises an expression construct encoding a fusion protein as described herein, e.g., on the same accessory plasmid or on a separate vector. In some embodiments, the host cell further provides any phage functions that are not contained in the selection phage, e.g., in the form of a helper phage. In some embodiments, the host cell provided further comprises an expression construct comprising a gene encoding a mutagenesis-inducing protein, for example, a mutagenesis plasmid as provided herein.

In some embodiments, modified viral vectors are used in continuous evolution processes as provided herein. In some embodiments, such modified viral vectors lack a gene required for the generation of infectious viral particles. In some such embodiments, a suitable host cell is a cell comprising the gene required for the generation of infectious viral particles, for example, under the control of a constitutive or a conditional promoter (e.g., in the form of an accessory plasmid, as described herein). In some embodiments, the viral vector used lacks a plurality of viral genes. In some such embodiments, a suitable host cell is a cell that comprises a helper construct providing the viral genes required for the generation of infectious viral particles. A cell is not required to actually support the life cycle of a viral vector used in the methods provided herein. For example, a cell comprising a gene required for the generation of infectious viral particles under the control of a conditional promoter may not support the life cycle of a viral vector that does not comprise a gene of interest able to activate the promoter, but it is still a suitable host cell for such a viral vector.

In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/viral vector combinations will be readily apparent to those of skill in the art.

In some embodiments, the viral vector is a phage and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, and XL1-Blue MRF′. These strain names are art recognized and the genotype of these strains has been well characterized. It should be understood that the above strains are exemplary only and that the invention is not limited in this respect.

In some PACE embodiments, for example, in embodiments employing an M13 selection phage, the host cells are E. coli cells expressing the Fertility factor, also commonly referred to as the F factor, sex factor, or F-plasmid. The F-factor is a bacterial DNA sequence that allows a bacterium to produce a sex pilus necessary for conjugation and is essential for the infection of E. coli cells with certain phage, for example, with M13 phage. For example, in some embodiments, the host cells for M13-PACE are of the genotype F′proA⁺B⁺A(lacIZY) zzf::Tn10(TetR)/endA1 recA1 galE15 galK16 nupG rpsL ΔlacIZYA araD139 Δ(ara,leu)7697 mcrA Δ(mrr-hsdRMS-mcrBC) proBA::pir116 λ.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### EXAMPLE

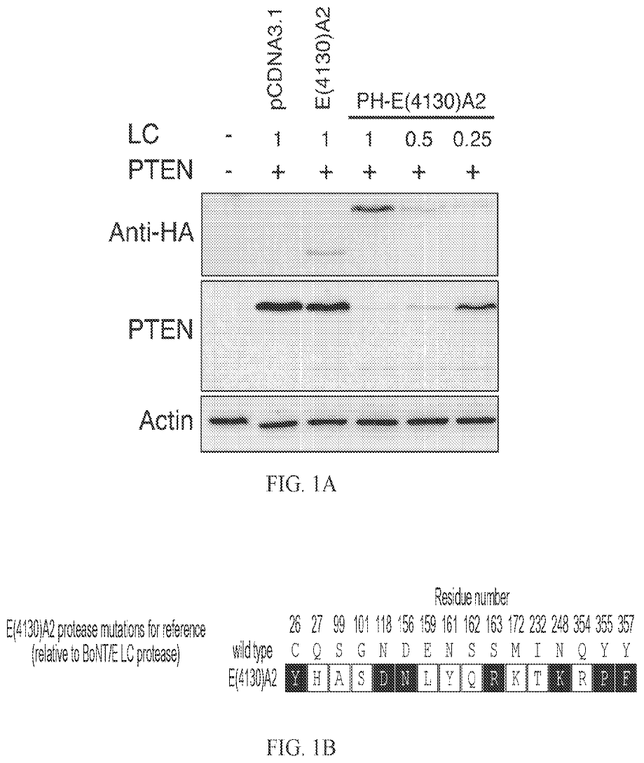

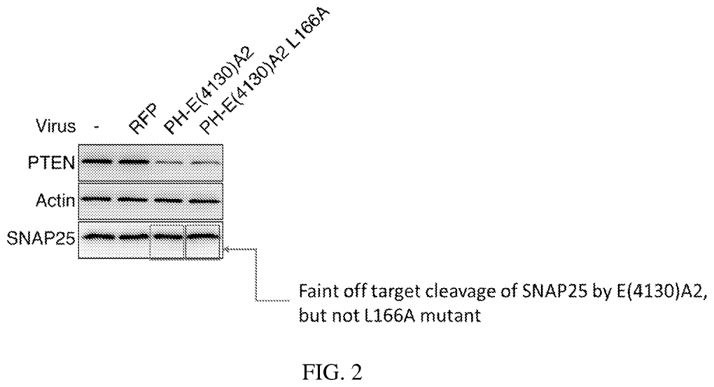

A PACE-evolved, PTEN-cleaving protease, “E(4130)A2”, was assessed by co-transfecting plasmids expressing both the protease and target PTEN into HEK293T cells. Since PTEN is trafficked to the plasma membrane in cells, a pleckstrin homology (PH) domain was fused to the N-terminus of the evolved protease to promote co-localization of the protease with its intended substrate. This modification generated “PH-E(4130)A2”, which performed highly efficient cleavage of PTEN when transfected into HEK293T cells (FIGS. 1A-1B). In addition, cells were transduced with lentivirus encoding PH-E(4130)A2, and cleavage of both PTEN and SNAP25 was examined by Western blot. The transduced cells show substantial PTEN cleavage with minimal off-target cleavage of the native BoNT/E substrate SNAP25 (FIG. 2). This selectivity was further enhanced by introducing the L166A mutation (L167A, relative to SEQ ID NO.: 1), which has been observed to reduce SNAP protein cleavage by BoNTs, into PH-E(4130)A2. These results indicate that the activity of proteases emerging from PACE selections can support efficient and selective targeted proteolysis in mammalian cells.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

### REPRESENTATIVE SEQUENCES

>Wild type Botulinum Neurotoxin E (BoNT/E) amino acid sequence (SEQ ID NO.: 1)
MPKINSFNYNDPVNDRTILYIKPGGCQEFYKSFNIMKNIWIIPERNVIGTTPQDFHPPTSL
KNGDSSYYDPNYLQSDEEKDRFLKIVTKIFNRINNNLSGGILLEELSKANPYLGNDNTPD
NQFHIGDASAVEIKFSNGSQHILLPNVIIMGAEPDLFETNSSNISLRNNYMPSNHGFGSIAI
VTFSPEYSFRFNDNSINEFIQDPALTLMHELIHSLHGLYGAKGITTTCIITQQQNPLITNRK
GINIEEFLTFGGNDLNIITVAQYNDIYTNLLNDYRKIASKLSKVQVSNPQLNPYKDIFQEK
YGLDKDASGIYSVNINKFDDILKKLYSFTEFDLATKFQVKCRETYIGQYKYFKLSNLLN
DSIYNISEGYNINNLKVNFRGQNANLNPRIIKPITGRGLVKKIIRF
>Human phospholipase C delta 1 (PLC81) pleckstrin homology (PH) domain amino acid
sequence (SEQ ID NO.: 2)
MDSGRDFLTLHGLQDDEDLQALLKGSQLLKVKSSSWRRERFYKLQEDCKTIWQESRKV
MRTPESQLFSIEDIQEVRMGHRTEGLEKFARDVPEDRCFSIVFKDQRNTLDLIAPSPADA
QHWVLGLHKIIHHSGSMDQRQKLQHWIHSCLRKADKNKDNKMSFKELQNFLK
>BoNT/E (4130)A2 variant amino acid sequence (SEQ ID NO: 3)
MPKINSFNYNDPVNDRTILYIKPGGYHEFYKSFNIMKNIWIIPERNVIGTTPQDFHPPTSL
KNGDSSYYDPNYLQSDEEKDRFLKIVTKIFNRINNNLAGSILLEELSKANPYLGNDDTPD
NQFHIGDASAVEIKFSNGSQHILLPNVIIMGAEPNLFLTYQRNISLRNNYKPSNHGFGSIAI
VTFSPEYSFRFNDNSINEFIQDPALTLMHELIHSLHGLYGAKGITTTCTITQQQNPLITNRK
GIKIEEFLTFGGNDLNIITVAQYNDIYTNLLNDYRKIASKLSKVQVSNPQLNPYKDIFQEK
YGLDKDASGIYSVNINKFDDILKKLYSFTEFDLATKFQVKCRETYIGRPKFFKLSNLLND
SIYNISEGYNINNLKVNFRGQNANLNPRIIKPITGRGLVKKIIRF
>BoNT/E (4130)A2-L166A variant amino acid sequence (SEQ ID NO: 4)
MPKINSFNYNDPVNDRTILYIKPGGYHEFYKSFNIMKNIWIIPERNVIGTTPQDFHPPTSL
KNGDSSYYDPNYLQSDEEKDRFLKIVTKIFNRINNNLAGSILLEELSKANPYLGNDDTPD
NQFHIGDASAVEIKFSNGSQHILLPNVIIMGAEPNLFLTYQRNISLRNNYKPSNHGFGSIAI
VTFSPEYSFRFNDNSINEFIQDPALTLMHELIHSLHGLYGAKGITTTCTITQQQNPLITNRK
GIKIEEFLTFGGNDLNIITVAQYNDIYTNLLNDYRKIASKLSKVQVSNPQLNPYKDIFQEK
YGLDKDASGIYSVNINKFDDILKKLYSFTEFDLATKFQVKCRETYIGRPKFFKLSNLLND
SIYNISEGYNINNLKVNFRGQNANLNPRIIKPITGRGLVKKIIRF
>PH-BoNT/E (4130)A2 fusion protein amino acid sequence (SEQ ID NO.: 5)
MDSGRDFLTLHGLQDDEDLQALLKGSQLLKVKSSSWRRERFYKLQEDCKTIWQESRKV
MRTPESQLFSIEDIQEVRMGHRTEGLEKFARDVPEDRCFSIVFKDQRNTLDLIAPSPADA
QHWVLGLHKIIHHSGSMDQRQKLQHWIHSCLRKADKNKDNKMSFKELQNFLKGGGGS
MPKINSFNYNDPVNDRTILYIKPGGYHEFYKSFNIMKNIWIIPERNVIGTTPQDFHPPTSL
KNGDSSYYDPNYLQSDEEKDRFLKIVTKIFNRINNNLAGSILLEELSKANPYLGNDDTPD
NQFHIGDASAVEIKFSNGSQHILLPNVIIMGAEPNLFLTYQRNISLRNNYKPSNHGFGSIAI
VTFSPEYSFRFNDNSINEFIQDPALTLMHELIHSLHGLYGAKGITTTCTITQQQNPLITNRK
GIKIEEFLTFGGNDLNIITVAQYNDIYTNLLNDYRKIASKLSKVQVSNPQLNPYKDIFQEK
YGLDKDASGIYSVNINKFDDILKKLYSFTEFDLATKFQVKCRETYIGRPKFFKLSNLLND
SIYNISEGYNINNLKVNFRGQNANLNPRIIKPITGRGLVKKIIRF
>PH-BoNT/E (4130)A2-L166A fusion protein amino acid sequence (SEQ ID NO.: 6)
MDSGRDFLTLHGLQDDEDLQALLKGSQLLKVKSSSWRRERFYKLQEDCKTIWQESRKV
MRTPESQLFSIEDIQEVRMGHRTEGLEKFARDVPEDRCESIVFKDQRNTLDLIAPSPADA
QHWVLGLHKIIHHSGSMDQRQKLQHWIHSCLRKADKNKDNKMSFKELQNFLKGGGGS
MPKINSFNYNDPVNDRTILYIKPGGYHEFYKSFNIMKNIWIIPERNVIGTTPQDFHPPTSL
KNGDSSYYDPNYLQSDEEKDRFLKIVTKIFNRINNNLAGSILLEELSKANPYLGNDDTPD
NQFHIGDASAVEIKFSNGSQHILLPNVIIMGAEPNLFLTYQRNISARNNYKPSNHGFGSIAI
VTFSPEYSFRFNDNSINEFIQDPALTLMHELIHSLHGLYGAKGITTTCTITQQQNPLITNRK
GIKIEEFLTFGGNDLNIITVAQYNDIYTNLLNDYRKIASKLSKVQVSNPQLNPYKDIFQEK
YGLDKDASGIYSVNINKFDDILKKLYSFTEFDLATKFQVKCRETYIGRPKFFKLSNLLND
SIYNISEGYNINNLKVNFRGQNANLNPRIIKPITGRGLVKKIIRF
>Human pleckstrin homology (PH) domain nucleic acid sequence (SEQ ID NO.: 7)
ATGGACTCGGGCCGGGACTTCCTGACCCTGCACGGCCTACAGGATGATGAGGATCT
ACAGGCGCTGCTGAAGGGCAGCCAGCTCCTGAAGGTGAAGTCCAGCTCATGGAGGA
GAGAGCGCTTCTACAAGTTGCAGGAGGACTGCAAGACCATCTGGCAGGAGTCCCGC
AAGGTCATGCGGACCCCGGAGTCCCAGCTGTTCTCCATCGAGGACATTCAGGAGGT
GCGAATGGGGCACCGCACGGAGGGTCTGGAGAAGTTCGCCCGTGATGTGCCCGAGG
ACCGCTGCTTCTCCATTGTCTTCAAGGACCAGCGCAATACACTAGACCTCATCGCCC
CATCGCCAGCTGATGCCCAGCACTGGGTGCTGGGGCTGCACAAGATCATCCACCAC
TCAGGCTCCATGGACCAGCGTCAGAAGCTACAGCACTGGATTCACTCCTGCTTGCG
AAAAGCTGACAAAAACAAGGACAACAAGATGAGCTTCAAGGAGCTGCAGAACTTC
CTGAAG
>BoNT/E (4130)A2 variant nucleic acid sequence (SEQ ID NO: 8)
ATGCCAAAAATCAACAGCTTTAATTACAATGACCCTGTAAACGATCGTACCATCCTA
TACATAAAGCCGGGTGGGTATCACGAGTTCTACAAATCTTTCAATATTATGAAGAAT
ATATGGATTATACCTGAGCGTAACGTTATTGGTACGACACCGCAAGATTTTCATCCA
CCTACTTCGTTGAAGAACGGTGACTCTTCCTATTACGACCCCAATTATCTCCAGTCG
GATGAAGAGAAGGACAGATTCCTTAAAATAGTAACCAAAATCTTTAACAGGATTAA
TAACAATCTAGCCGGAAGTATTTTGCTTGAAGAGCTTAGTAAAGCTAATCCTTACCT
AGGTAACGATGATACACCAGACAACCAGTTTCATATAGGCGATGCATCCGCCGTGG
AAATCAAATTTAGCAATGGATCACAGCATATTCTCTTGCCCAACGTTATTATAATGG
GGGCGGAACCAAATTTATTTTTGACATATCAGAGAAATATTAGCCTGAGAAATAAC
TATAAGCCGTCAAACCATGGGTTCGGTAGCATAGCAATCGTTACTTTTTCTCCCGAA
TACAGTTTTCGCTTCAATGATAATAGTATAAATGAGTTTATCCAAGACCCCGCACTC
ACGCTTATGCACGAACTCATACACTCTTTACACGGCCTGTATGGCGCTAAGGGGATA
ACCACTACGTGTACCATTACTCAGCAACAGAACCCATTGATAACGAACAGGAAGGG
CATTAAAATCGAGGAATTTCTTACATTTGGAGGCAACGATCTGAACATTATAACTGT
CGCACAGTACAATGACATCTATACCAACTTACTAAATGATTATAGAAAAATCGCTTC
TAAGTTATCCAAGGTTCAAGTCTCAAACCCTCAACTGAATCCGTATAAGGACATATT
CCAAGAAAAATATGGATTAGACAAAGACGCGTCAGGAATCTATTCGGTAAACATTA
ACAAATTCGACGATATTTTGAAGAAACTTTACAGCTTCACGGAGTTCGACTTGGCCA
CCAAATTCCAGGTCAAATGCCGAGAGACATACATCGGACGGCCTAAGTTTTTTAAG
CTGTCGAATCTCCTGAATGATTCCATATACAACATTAGTGAGGGTTACAATATAAAT
AACCTAAAGGTGAATTTCCGAGGCCAAAACGCCAACCTAAATCCGCGTATCATTAA
ACCCATCACAGGACGGGGGTTAGTGAAGAAAATAATCCGGTTT
>BoNT/E (4130)A2-L166A variant nucleic acid sequence (SEQ ID NO: 9)
ATGCCAAAAATCAACAGCTTTAATTACAATGACCCTGTAAACGATCGTACCATCCTA
TACATAAAGCCGGGTGGGTATCACGAGTTCTACAAATCTTTCAATATTATGAAGAAT
ATATGGATTATACCTGAGCGTAACGTTATTGGTACGACACCGCAAGATTTTCATCCA
CCTACTTCGTTGAAGAACGGTGACTCTTCCTATTACGACCCCAATTATCTCCAGTCG
GATGAAGAGAAGGACAGATTCCTTAAAATAGTAACCAAAATCTTTAACAGGATTAA
TAACAATCTAGCCGGAAGTATTTTGCTTGAAGAGCTTAGTAAAGCTAATCCTTACCT
AGGTAACGATGATACACCAGACAACCAGTTTCATATAGGCGATGCATCCGCCGTGG
AAATCAAATTTAGCAATGGATCACAGCATATTCTCTTGCCCAACGTTATTATAATGG
GGGCGGAACCAAATTTATTTTTGACATATCAGAGAAATATTAGCGCCAGAAATAAC
TATAAGCCGTCAAACCATGGGTTCGGTAGCATAGCAATCGTTACTTTTTCTCCCGAA
TACAGTTTTCGCTTCAATGATAATAGTATAAATGAGTTTATCCAAGACCCCGCACTC
ACGCTTATGCACGAACTCATACACTCTTTACACGGCCTGTATGGCGCTAAGGGGATA
ACCACTACGTGTACCATTACTCAGCAACAGAACCCATTGATAACGAACAGGAAGGG
CATTAAAATCGAGGAATTTCTTACATTTGGAGGCAACGATCTGAACATTATAACTGT
CGCACAGTACAATGACATCTATACCAACTTACTAAATGATTATAGAAAAATCGCTTC
TAAGTTATCCAAGGTTCAAGTCTCAAACCCTCAACTGAATCCGTATAAGGACATATT
CCAAGAAAAATATGGATTAGACAAAGACGCGTCAGGAATCTATTCGGTAAACATTA
ACAAATTCGACGATATTTTGAAGAAACTTTACAGCTTCACGGAGTTCGACTTGGCCA
CCAAATTCCAGGTCAAATGCCGAGAGACATACATCGGACGGCCTAAGTTTTTTAAG
CTGTCGAATCTCCTGAATGATTCCATATACAACATTAGTGAGGGTTACAATATAAAT
AACCTAAAGGTGAATTTCCGAGGCCAAAACGCCAACCTAAATCCGCGTATCATTAA
ACCCATCACAGGACGGGGGTTAGTGAAGAAAATAATCCGGTTT
>PH-BoNT/E (4130)A2 fusion protein nucleic acid sequence (SEQ ID NO.: 10)
ATGGACTCGGGCCGGGACTTCCTGACCCTGCACGGCCTACAGGATGATGAGGATCT
ACAGGCGCTGCTGAAGGGCAGCCAGCTCCTGAAGGTGAAGTCCAGCTCATGGAGGA
GAGAGCGCTTCTACAAGTTGCAGGAGGACTGCAAGACCATCTGGCAGGAGTCCCGC
AAGGTCATGCGGACCCCGGAGTCCCAGCTGTTCTCCATCGAGGACATTCAGGAGGT
GCGAATGGGGCACCGCACGGAGGGTCTGGAGAAGTTCGCCCGTGATGTGCCCGAGG
ACCGCTGCTTCTCCATTGTCTTCAAGGACCAGCGCAATACACTAGACCTCATCGCCC
CATCGCCAGCTGATGCCCAGCACTGGGTGCTGGGGCTGCACAAGATCATCCACCAC
TCAGGCTCCATGGACCAGCGTCAGAAGCTACAGCACTGGATTCACTCCTGCTTGCG
AAAAGCTGACAAAAACAAGGACAACAAGATGAGCTTCAAGGAGCTGCAGAACTTC
CTGAAGGGTGGTGGTGGTAGCATGCCAAAAATCAACAGCTTTAATTACAATGACCC
TGTAAACGATCGTACCATCCTATACATAAAGCCGGGTGGGTATCACGAGTTCTACA
AATCTTTCAATATTATGAAGAATATATGGATTATACCTGAGCGTAACGTTATTGGTA
CGACACCGCAAGATTTTCATCCACCTACTTCGTTGAAGAACGGTGACTCTTCCTATT
ACGACCCCAATTATCTCCAGTCGGATGAAGAGAAGGACAGATTCCTTAAAATAGTA
ACCAAAATCTTTAACAGGATTAATAACAATCTAGCCGGAAGTATTTTGCTTGAAGA
GCTTAGTAAAGCTAATCCTTACCTAGGTAACGATGATACACCAGACAACCAGTTTC
ATATAGGCGATGCATCCGCCGTGGAAATCAAATTTAGCAATGGATCACAGCATATT
CTCTTGCCCAACGTTATTATAATGGGGGCGGAACCAAATTTATTTTTGACATATCAG
AGAAATATTAGCCTGAGAAATAACTATAAGCCGTCAAACCATGGGTTCGGTAGCAT
AGCAATCGTTACTTTTTCTCCCGAATACAGTTTTCGCTTCAATGATAATAGTATAAA
TGAGTTTATCCAAGACCCCGCACTCACGCTTATGCACGAACTCATACACTCTTTACA
CGGCCTGTATGGCGCTAAGGGGATAACCACTACGTGTACCATTACTCAGCAACAGA
ACCCATTGATAACGAACAGGAAGGGCATTAAAATCGAGGAATTTCTTACATTTGGA
GGCAACGATCTGAACATTATAACTGTCGCACAGTACAATGACATCTATACCAACTT
ACTAAATGATTATAGAAAAATCGCTTCTAAGTTATCCAAGGTTCAAGTCTCAAACCC
TCAACTGAATCCGTATAAGGACATATTCCAAGAAAAATATGGATTAGACAAAGACG
CGTCAGGAATCTATTCGGTAAACATTAACAAATTCGACGATATTTTGAAGAAACTTT
ACAGCTTCACGGAGTTCGACTTGGCCACCAAATTCCAGGTCAAATGCCGAGAGACA
TACATCGGACGGCCTAAGTTTTTTAAGCTGTCGAATCTCCTGAATGATTCCATATAC
AACATTAGTGAGGGTTACAATATAAATAACCTAAAGGTGAATTTCCGAGGCCAAAA
CGCCAACCTAAATCCGCGTATCATTAAACCCATCACAGGACGGGGGTTAGTGAAGA
AAATAATCCGGTTT
>PH-BoNT/E (4130)A2-L166A fusion protein nucleic acid sequence (SEQ ID NO.: 11)
ATGGACTCGGGCCGGGACTTCCTGACCCTGCACGGCCTACAGGATGATGAGGATCT
ACAGGCGCTGCTGAAGGGCAGCCAGCTCCTGAAGGTGAAGTCCAGCTCATGGAGGA
GAGAGCGCTTCTACAAGTTGCAGGAGGACTGCAAGACCATCTGGCAGGAGTCCCGC
AAGGTCATGCGGACCCCGGAGTCCCAGCTGTTCTCCATCGAGGACATTCAGGAGGT
GCGAATGGGGCACCGCACGGAGGGTCTGGAGAAGTTCGCCCGTGATGTGCCCGAGG
ACCGCTGCTTCTCCATTGTCTTCAAGGACCAGCGCAATACACTAGACCTCATCGCCC
CATCGCCAGCTGATGCCCAGCACTGGGTGCTGGGGCTGCACAAGATCATCCACCAC
TCAGGCTCCATGGACCAGCGTCAGAAGCTACAGCACTGGATTCACTCCTGCTTGCG
AAAAGCTGACAAAAACAAGGACAACAAGATGAGCTTCAAGGAGCTGCAGAACTTC
CTGAAGGGTGGTGGTGGTAGCATGCCAAAAATCAACAGCTTTAATTACAATGACCC
TGTAAACGATCGTACCATCCTATACATAAAGCCGGGTGGGTATCACGAGTTCTACA
AATCTTTCAATATTATGAAGAATATATGGATTATACCTGAGCGTAACGTTATTGGTA
CGACACCGCAAGATTTTCATCCACCTACTTCGTTGAAGAACGGTGACTCTTCCTATT
ACGACCCCAATTATCTCCAGTCGGATGAAGAGAAGGACAGATTCCTTAAAATAGTA
ACCAAAATCTTTAACAGGATTAATAACAATCTAGCCGGAAGTATTTTGCTTGAAGA
GCTTAGTAAAGCTAATCCTTACCTAGGTAACGATGATACACCAGACAACCAGTTTC
ATATAGGCGATGCATCCGCCGTGGAAATCAAATTTAGCAATGGATCACAGCATATT
CTCTTGCCCAACGTTATTATAATGGGGGCGGAACCAAATTTATTTTTGACATATCAG
AGAAATATTAGCGCCAGAAATAACTATAAGCCGTCAAACCATGGGTTCGGTAGCAT
AGCAATCGTTACTTTTTCTCCCGAATACAGTTTTCGCTTCAATGATAATAGTATAAA
TGAGTTTATCCAAGACCCCGCACTCACGCTTATGCACGAACTCATACACTCTTTACA
CGGCCTGTATGGCGCTAAGGGGATAACCACTACGTGTACCATTACTCAGCAACAGA
ACCCATTGATAACGAACAGGAAGGGCATTAAAATCGAGGAATTTCTTACATTTGGA
GGCAACGATCTGAACATTATAACTGTCGCACAGTACAATGACATCTATACCAACTT
ACTAAATGATTATAGAAAAATCGCTTCTAAGTTATCCAAGGTTCAAGTCTCAAACCC
TCAACTGAATCCGTATAAGGACATATTCCAAGAAAAATATGGATTAGACAAAGACG
CGTCAGGAATCTATTCGGTAAACATTAACAAATTCGACGATATTTTGAAGAAACTTT
ACAGCTTCACGGAGTTCGACTTGGCCACCAAATTCCAGGTCAAATGCCGAGAGACA
TACATCGGACGGCCTAAGTTTTTTAAGCTGTCGAATCTCCTGAATGATTCCATATAC
AACATTAGTGAGGGTTACAATATAAATAACCTAAAGGTGAATTTCCGAGGCCAAAA
CGCCAACCTAAATCCGCGTATCATTAAACCCATCACAGGACGGGGGTTAGTGAAGA
AAATAATCCGGTTT
>Human PTEN amino acid sequence (SEQ ID NO.: 12)
MTAIIKEIVSRNKRRYQEDGFDLDLTYIYPNIIAMGFPAERLEGVYRNNIDDVVRFLDSK
HKNHYKIYNLCAERHYDTAKFNCRVAQYPFEDHNPPQLELIKPFCEDLDQWLSEDDNH
VAAIHCKAGKGRTGVMICAYLLHRGKFLKAQEALDFYGEVRTRDKKGVTIPSORRYVY
YYSYLLKNHLDYRPVALLFHKMMFETIPMFSGGTCNPQFVVCQLKVKIYSSNSGPTRRE
DKFMYFEFPQPLPVCGDIKVEFFHKQNKMLKKDKMFHFWVNTFFIPGPEETSEKVENGS
LCDQEIDSICSIERADNDKEYLVLTLTKNDLDKANKDKANRYFSPNFKVKLYFTKTVE
EPSNPEASSSTSVTPDVSDNEPDHYRYSDTTDSDPENEPFDEDQHTQITKV
>Minimized PTEN cleavage sequence (SEQ ID NO.: 13)
NGSLCDQEIDSICSIMEKAD
>BoNT E HCN Domain (SEQ ID NO.: 14)
CKNIVSVKGIRKSICIEINNGELFFVASENSYNDDNINTPKEIDDTVTSNNNYENDLDQVI
LNFNSESAPGLSDEKLNLTIQNDAYIPKYDSNGTSDIEQHDVNELNVFFYLDAQKVPEGE
NNVNLTSSIDTALLEQPKIYTFFSSEFINNVNKPVQAALFVSWIQQVLVDFTTEANQKST
VDKIADISIVVPYIGLALNIGNEAQKGNFKDALELLGAGILLEFEPELLIPTILVFTIKSFLG
SSDNKNKVIKAINNALKERDEKWKEVYSFIVSNWMTKINTQFNKRKEQMYQALQNQV
NAIKTIIESKYNSYTLEEKNELTNKYDIKQIENELNQKVSIAMNNIDRFLTESSISYLMKLI
NEVKINKLREYDENVKTYLLNYIIQHGSILGESQQELNSMVTDTLNNSIPFKLSSYTDDKI
LISYFNKFFKRIKS (BoNT E HC_{N}, translocation domain)
>BoNT E HCC Domain (SEQ ID NO.: 15)
SSVLNMRYKNDKYVDTSGYDSNININGDVYKYPTNKNQFEIYNDKLSEVNISQNDYIIY
DNKYKNFSISFWVRIPNYDNKIVNVNNEYTIINCMRDNNSGWKVSLNHNEIIWTLQDNA
GINQKLAFNYGNANGISDYINKWIFVTITNDRLGDSKLYINGNLIDQKSILNLGNIHVSD
NILFKIVNCSYTRYIGIRYFNIFDKELDETEIQTLYSNEPNTNILKDFWGNYLLYDKEYYL
LNVLKPNNFIDRRKDSTLSINNIRSTILLANRLYSGIKVKIQRVNNSSTNDNLVRKNDQV
YINFVASKTHLFPLYADTATTNKEKTIKISSSGNRFNQVVVMNSVGNNCTMNFKNNNG
NNIGLLGFKADTVVASTWYYTHMRDHTNSNGCFWNFISEEHGWQEK (BoNT E HC_{C},
Binding domain)
>SNAP25 substrate sequence (SEQ ID NO.: 16)
MAEDADMRNELEEMQRRADQLADESLESTRRMLQLVEESKDAGIRTLVMLDEQGEQL
ERIEEGMDQINKDMKEAEKNLTDLGKFCGLCVCPCNKLKSSDAYKKAWGNNQDGVV
ASQPARVVDEREQMAISGGFIRRVTNDARENEMDENLEQVSGIIGNLRHMALDMGNEI
DTQNRQIDRIMEKADSNKTRIDEANQRATKMLGSG
>SNAP25 substate amino acid sequence (SEQ ID NO.: 17)
RQIDRIMEKA
>Human cytohesin-1 PH domain amino acid sequence (SEQ ID NO.: 18)
NPDREGWLLKLGGGRVKTWKRRWFILTDNCLYYFEYTTDKEPRGIIPLENLSIREVEDS
KKPNCFELYIPDNKDQVIKACKTEADGRVVEGNHTVYRISAPTPEEKEEWIKCIKAAIS
>Human cytohesin-2 PH domain amino acid sequence (SEQ ID NO.: 19)
NPDREGWLLKLGGGRVKTWKRRWFILTDNCLYYFEYTTDKEPRGIIPLENLSIREVDDP
RKPNCFELYIPNNKGQLIKACKTEADGRVVEGNHMVYRISAPTQEEKDEWIKSIQAAVS
>Human cytohesin-3 PH domain amino acid sequence (SEQ ID NO.: 20)
NPDREGWLLKLGGGRVKTWKRRWFILTDNCLYYFEYTTDKEPRGIIPLENLSIREVEDP
RKPNCFELYNPSHKGQVIKACKTEADGRVVEGNHVVYRISAPSPEEKEEWMKSIKASIS
>Human tyrosine-protein kinase BTK PH domain amino acid sequence (SEQ ID NO.: 21)
AVILESIFLKRSQQKKKTSPLNFKKRLFLLTVHKLSYYEYDFERGRRGSKKGSIDVEKITC
VETVVPEKNPPPERQIPRRGEESSEMEQISIIERFPYPFQVVYDEGPLYVFSPTEELRKRWI
HQLKNVIR
>Human PTEN nucleic acid sequence (SEQ ID NO.: 22)
ATGACAGCCATCATCAAAGAGATCGTTAGCAGAAACAAAAGGAGATATCAAGAGG
ATGGATTCGACTTAGACTTGACCTATATTTATCCAAACATTATTGCTATGGGATTTC
CTGCAGAAAGACTTGAAGGCGTATACAGGAACAATATTGATGATGTAGTAAGGTTT
TTGGATTCAAAGCATAAAAACCATTACAAGATATACAATCTTTGTGCTGAAAGACA
TTATGACACCGCCAAATTTAATTGCAGAGTTGCACAATATCCTTTTGAAGACCATAA
CCCACCACAGCTAGAACTTATCAAACCCTTTTGTGAAGATCTTGACCAATGGCTAAG
TGAAGATGACAATCATGTTGCAGCAATTCACTGTAAAGCTGGAAAGGGACGAACTG
GTGTAATGATATGTGCATATTTATTACATCGGGGCAAATTTTTAAAGGCACAAGAG
GCCCTAGATTTCTATGGGGAAGTAAGGACCAGAGACAAAAAGGGAGTAACTATTCC
CAGTCAGAGGCGCTATGTGTATTATTATAGCTACCTGTTAAAGAATCATCTGGATTA
TAGACCAGTGGCACTGTTGTTTCACAAGATGATGTTTGAAACTATTCCAATGTTCAG
TGGCGGAACTTGCAATCCTCAGTTTGTGGTCTGCCAGCTAAAGGTGAAGATATATTC
CTCCAATTCAGGACCCACACGACGGGAAGACAAGTTCATGTACTTTGAGTTCCCTCA
GCCGTTACCTGTGTGTGGTGATATCAAAGTAGAGTTCTTCCACAAACAGAACAAGA
TGCTAAAAAAGGACAAAATGTTTCACTTTTGGGTAAATACATTCTTCATACCAGGAC
CAGAGGAAACCTCAGAAAAAGTAGAAAATGGAAGTCTATGTGATCAAGAAATCGA
TAGCATTTGCAGTATAGAGCGTGCAGATAATGACAAGGAATATCTAGTACTTACTTT
AACAAAAAATGATCTTGACAAAGCAAATAAAGACAAAGCCAACCGATACTTTTCTC
CAAATTTTAAGGTGAAGCTGTACTTCACAAAAACAGTAGAGGAGCCGTCAAATCCA
GAGGCTAGCAGTTCAACTTCTGTAACACCAGATGTTAGTGACAATGAACCTGATCA
TTATAGATATTCTGACACCACTGACTCTGATCCAGAGAATGAACCTTTTGATGAAGA
TCAGCATACACAAATTACAAAAGTC
>Human cytohesin-1 PH domain nucleic acid sequence (SEQ ID NO.: 23)
AATCCAGACCGAGAAGGCTGGCTATTGAAACTCGGAGGTGGCAGGGTAAAGACTTG
GAAGAGACGCTGGTTCATTCTGACTGACAACTGCCTTTACTACTTTGAGTATACCAC
GGATAAGGAGCCCCGTGGAATCATCCCTTTAGAGAATCTGAGTATCCGGGAAGTGG
AGGACTCCAAAAAACCAAACTGCTTTGAGCTTTATATCCCCGACAATAAAGACCAA
GTTATCAAGGCCTGCAAGACCGAGGCTGACGGGCGGGTGGTGGAGGGGAACCACA
CTGTTTACCGGATCTCAGCTCCGACGCCCGAGGAGAAGGAGGAGTGGATTAAGTGC
ATTAAAGCAGCCATCAGC
>Human cytohesin-2 PH domain nucleic acid sequence (SEQ ID NO.: 24)
AACCCGGACCGGGAGGGCTGGCTCCTGAAGCTGGGGGGGGGGGGGTGAAGACGT
GGAAGCGGCGCTGGTTTATCCTCACAGACAACTGCCTCTACTACTTTGAGTACACCA
CGGACAAGGAGCCCCGAGGAATCATCCCCCTGGAGAATCTGAGCATCCGAGAGGTG
GACGACCCCCGGAAACCGAACTGCTTTGAACTTTACATCCCCAACAACAAGGGGCA
GCTCATCAAAGCCTGCAAAACTGAGGCGGACGGCCGAGTGGTGGAGGGAAACCAC
ATGGTGTACCGGATCTCGGCCCCCACGCAGGAGGAGAAGGACGAGTGGATCAAGTC
CATCCAGGCGGCTGTGAGT
>Human cytohesin-3 PH domain nucleic acid sequence (SEQ ID NO.: 25)
AACCCCGACCGCGAGGGCTGGCTCCTGAAGCTGGGAGGGGGCCGTGTGAAGACCTG
GAAGCGCCGGTGGTTCATCCTGACCGATAACTGCCTCTATTACTTTGAATACACAAC
AGATAAGGAGCCCAGGGGAATCATCCCGTTGGAAAACCTCAGCATCAGGGAGGTG
GAGGACCCCCGGAAACCCAACTGTTTTGAGCTCTACAATCCCAGCCACAAAGGGCA
GGTCATCAAGGCCTGTAAGACTGAGGCCGACGGCCGCGTGGTAGAGGGGAACCAT
GTGGTGTACCGGATCTCAGCCCCGAGCCCGGAGGAGAAGGAGGAGTGGATGAAAT
CCATCAAAGCCAGTATCAGC
>Human tyrosine-protein kinase BTK PH domain nucleic acid sequence (SEQ ID NO.: 26)
GCAGTGATTCTGGAGAGCATCTTTCTGAAGCGATCCCAACAGAAAAAGAAAACATC
ACCTCTAAACTTCAAGAAGCGCCTGTTTCTCTTGACCGTGCACAAACTCTCCTACTA
TGAGTATGACTTTGAACGTGGGAGAAGAGGCAGTAAGAAGGGTTCAATAGATGTTG
AGAAGATCACTTGTGTTGAAACAGTGGTTCCTGAAAAAAATCCTCCTCCAGAAAGA
CAGATTCCGAGAAGAGGTGAAGAGTCCAGTGAAATGGAGCAAATTTCAATCATTGA
AAGGTTCCCTTATCCCTTCCAGGTTGTATATGATGAAGGGCCTCTCTACGTCTTCTCC
CCAACTGAAGAACTAAGGAAGCGGTGGATTCACCAGCTCAAAAACGTAATCCGG

What is claimed is:

1. A fusion protein comprising:
(i) a pleckstrin homology (PH) domain; and
(ii) a BoNT/E protease light chain having at least 80% sequence identity to SEQ ID NO.: 1.

2. The fusion protein of claim 1, wherein the PH domain is a human phospholipase C delta (PLCδ) PH domain.

3. The fusion protein of claim 1 or 2, wherein the PH domain has an amino acid sequence that is at least 80% identical to the sequence set forth in SEQ ID NO.: 2.

4. The fusion protein of any one of claims 1 to 3, wherein the BoNT/E protease light chain comprises an amino acid substitution in at least one of the following positions relative to SEQ ID NO. 1: C26, Q27, E28, 135, G49, H56, H56, S99, G101, N118, D156, E159, N161, S162, S163, S166, L167, M172, I203, I232, T242, R244, N248, I262, I263, A313, I316, G353, Q354, Y355, Y357, K359, N365, S367, N390, G403, or L404.

5. The fusion protein of any one of claims 1 to 4, wherein the BoNT/E protease light chain comprises at least one of the following amino acid substitutions relative to SEQ ID NO.: 1: C26Y, Q27H, E28K, I35V, G49S, H56L, H56Y, S99A, S99T, G101S, N118D, D156N, E159L, N161Y, S162Q, S163R, S166R, M172K, I203V, I232T, T242A, R244V, N248K, I262T, I263V, A313V, I316T, G353E, Q354R, Q354W, Y355P, Y355H, Y357F, K359R, N365S, S367F, N390D, G403E, or L404*.

6. The fusion protein of any one of claims 1 to 5, wherein the BoNT/E protease comprises the following amino acid substitutions relative to SEQ ID NO.: 1: C26Y, Q27H, S99A, G101S, N118D, D156N, E159L, N161Y, S162Q, S163R, L167A, M172K, I232T, N248K, Q354R, Y355P, and Y357F.

7. The fusion protein of any one of claims 1 to 6, wherein the fusion protein has at least 80% sequence identity (e.g., at least 80%, at least 85%, at least 90%, at least 95%, at least 96%, at least 97%, at least 98%, at least 99%, at least 99.5%, at least 99.9%, or more) to SEQ ID NO.: 5 or 6.

8. The fusion protein of any one of claims 1 to 7, wherein the fusion protein comprises or consists of the amino acid sequence set forth in SEQ ID NO.: 5 or 6.

9. The fusion protein of any one of claims 1 to 8, wherein the PH domain is positioned N-terminal relative to the BoNT/E protease light chain.

10. The fusion protein of any one of claims 1 to 9, wherein the PH domain and the BoNT/E protease light chain are directly connected.

11. The fusion protein of any one of claims 1 to 9 further comprising a linker.

12. The fusion protein of claim 11, wherein the linker comprises a peptide linker.

13. The fusion protein of claim 12, wherein the peptide linker comprises a glycine-rich linker, a proline-rich linker, glycine/serine-rich linker, and/or alanine/glutamic acid-rich linker.

14. The fusion protein of any one of claims 1 to 13, wherein the BoNT/E protease light chain is catalytically active.

15. The fusion protein of any one of claims 1 to 14, wherein the BoNT/E protease light chain is capable of cleaving a non-canonical BoNT/E substrate.

16. The fusion protein of claim 15, wherein the non-canonical BoNT/E substrate is a Phosphatase and tensin homolog (PTEN) protein.

17. The fusion protein of claim 16, wherein the PTEN protein comprises an amino acid sequence that is at least 70%, 80%, 90%, 95%, or 99% identical to the amino acid sequence set forth in SEQ ID NO.: 12 or 13.

18. The fusion protein of any one of claims 1 to 17, wherein the BoNT/E protease light chain does not cleave a SNAP protein.

19. The fusion protein of claim 18, wherein the BoNT/E protease light chain does not cleave SNAP25.

20. The fusion protein of claim 19, wherein the SNAP25 comprises the sequence set forth in SEQ ID NO: 16 or 17.

21. An isolated nucleic acid encoding the fusion protein of any one of claims 1 to 20.

22. The isolated nucleic acid of claim 21 having at least 60% 70%, 80%, 90%, 95%, or 99% or more to a nucleic acid sequence set forth in SEQ ID NO.: 10 or 11.

23. The isolated nucleic acid of claim 21 or claim 22, wherein the isolated nucleic acid comprises or consists of the nucleic acid sequence set forth in SEQ ID NO.: 10 or 11.

24. The isolated nucleic acid of any one of claims 21 to 23, wherein the nucleic acid sequence encoding the fusion protein is codon-optimized for expression in mammalian cells.

25. A vector comprising the isolated nucleic acid of any one of claims 21 to 24.

26. The vector of claim 25, wherein the vector is a plasmid or a viral vector.

27. The vector of claim 26, wherein the viral vector is a lentiviral vector.

28. A host cell comprising the fusion protein of any one of claims 1 to 17, the isolated nucleic acid of any one of claims 18 to 20, or the vector of any one of claims 21 to 23.

29. The host cell of claim 28, wherein the cell is a mammalian cell.

30. A method of cleaving an intracellular protein, the method comprising delivering to a cell the fusion protein of any one of claims 1 to 17, the isolated nucleic acid of any one of claims 21 to 24, or the vector of any one of claims 25 to 27, whereby the fusion protein contacts and cleaves the intracellular protein in the cell.

31. The method of claim 30, wherein the intracellular protein is a PTEN protein.

32. The method of claim 30 or 31, wherein the cell is a mammalian cell.

33. The method of any one of claims 30 to 32, wherein the cell membrane is intact.

34. The method of any one of claims 30 to 33, wherein the intracellular protein is cleaved in the plasma membrane of the cell.

35. Use of the fusion protein of any one of claims 1 to 20, the isolated nucleic acid of any one of claims 21 to 24, or the vector of any one of claims 25 to 27 in reducing PTEN activity or the amount of functional PTEN in a cell or subject.

36. The use of claim 35, wherein the cell is a mammalian cell.

37. The use of claim 36, wherein the cell is a human cell.

38. The use of any one of claims 35 to 37, wherein the cell is intact.

39. The use of any one of claims 35 to 38, wherein the cell is in a subject.

40. The use of claim 31, wherein the subject is a mammal.

41. The use of claim 40, wherein the subject is a human.

42. The use of any one of claims 31 to 41, wherein the cell or subject is characterized as having PTEN activity or expression that is higher than a normal healthy cell or subject.


SEQUENCE LISTING (28 sequences)

SEQ ID NO: 1 - protein, 411 aa, Unknown
  Botulinum Neurotoxin E
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQHILLPNVIIMGAEPDLFETNSSNISLRNNYMPSNHGFGSIAIVTFSPEYSFRFNDNSINEFIQDPALTLMHELIHSLHGLYGAKGITTTCIITQQQNPLITNRKGINIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGQYKYFKLSNLLNDSIYNISEGYNINNLKVNFRGQNANLNPRIIKPITGRGLVKKIIRF

SEQ ID NO: 2 - protein, 170 aa, Homo sapiens
MDSGRDFLTLHGLQDDEDLQALLKGSQLLKVKSSSWRRERFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLK

SEQ ID NO: 3 - protein, 411 aa, Artificial Sequence
  Synthetic
MPKINSFNYNDPVNDRTILYIKPGGYHEFYKSFNIMKNIWIIPERNVIGTTPQDFHPPTSLKNGDSSYYDPNYLQSDEEKDRFLKIVTKIFNRINNNLAGSILLEELSKANPYLGNDDTPDNQFHIGDASAVEIKFSNGSQHILLPNVIIMGAEPNLFLTYQRNISLRNNYKPSNHGFGSIAIVTFSPEYSFRFNDNSINEFIQDPALTLMHELIHSLHGLYGAKGITTTCTITQQQNPLITNRKGIKIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGRPKFFKLSNLLNDSIYNISEGYNINNLKVNFRGQNANLNPRIIKPITGRGLVKKIIRF

SEQ ID NO: 4 - protein, 411 aa, Artificial Sequence; identical to SEQ ID NO: 3
  Synthetic

SEQ ID NO: 5 - protein, 586 aa, Artificial Sequence
  Synthetic
MDSGRDFLTLHGLQDDEDLQALLKGSQLLKVKSSSWRRERFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLKGGGGSMPKINSFNYNDPVNDRTILYIKPGGYHEFYKSFNIMKNIWIIPERNVIGTTPQDFHPPTSLKNGDSSYYDPNYLQSDEEKDRFLKIVTKIFNRINNNLAGSILLEELSKANPYLGNDDTPDNQFHIGDASAVEIKFSNGSQHILLPNVIIMGAEPNLFLTYQRNISLRNNYKPSNHGFGSIAIVTFSPEYSFRFNDNSINEFIQDPALTLMHELIHSLHGLYGAKGITTTCTITQQQNPLITNRKGIKIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGRPKFFKLSNLLNDSIYNISEGYNINNLKVNFRGQNANLNPRIIKPITGRGLVKKIIRF

SEQ ID NO: 6 - protein, 586 aa, Artificial Sequence
  Synthetic
MDSGRDFLTLHGLQDDEDLQALLKGSQLLKVKSSSWRRERFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLKGGGGSMPKINSFNYNDPVNDRTILYIKPGGYHEFYKSFNIMKNIWIIPERNVIGTTPQDFHPPTSLKNGDSSYYDPNYLQSDEEKDRFLKIVTKIFNRINNNLAGSILLEELSKANPYLGNDDTPDNQFHIGDASAVEIKFSNGSQHILLPNVIIMGAEPNLFLTYQRNISARNNYKPSNHGFGSIAIVTFSPEYSFRFNDNSINEFIQDPALTLMHELIHSLHGLYGAKGITTTCTITQQQNPLITNRKGIKIEEFLTFGGNDLNIITVAQYNDIYTNLLNDYRKIASKLSKVQVSNPQLNPYKDIFQEKYGLDKDASGIYSVNINKFDDILKKLYSFTEFDLATKFQVKCRETYIGRPKFFKLSNLLNDSIYNISEGYNINNLKVNFRGQNANLNPRIIKPITGRGLVKKIIRF

SEQ ID NO: 7 - protein, 510 aa, Artificial Sequence
  Synthetic
ATGGACTCGGGCCGGGACTTCCTGACCCTGCACGGCCTACAGGATGATGAGGATCTACAGGCGCTGCTGAAGGGCAGCCAGCTCCTGAAGGTGAAGTCCAGCTCATGGAGGAGAGAGCGCTTCTACAAGTTGCAGGAGGACTGCAAGACCATCTGGCAGGAGTCCCGCAAGGTCATGCGGACCCCGGAGTCCCAGCTGTTCTCCATCGAGGACATTCAGGAGGTGCGAATGGGGCACCGCACGGAGGGTCTGGAGAAGTTCGCCCGTGATGTGCCCGAGGACCGCTGCTTCTCCATTGTCTTCAAGGACCAGCGCAATACACTAGACCTCATCGCCCCATCGCCAGCTGATGCCCAGCACTGGGTGCTGGGGCTGCACAAGATCATCCACCACTCAGGCTCCATGGACCAGCGTCAGAAGCTACAGCACTGGATTCACTCCTGCTTGCGAAAAGCTGACAAAAACAAGGACAACAAGATGAGCTTCAAGGAGCTGCAGAACTTCCTGAAG

SEQ ID NO: 8 - protein, 1233 aa, Artificial Sequence
  Synthetic
ATGCCAAAAATCAACAGCTTTAATTACAATGACCCTGTAAACGATCGTACCATCCTATACATAAAGCCGGGTGGGTATCACGAGTTCTACAAATCTTTCAATATTATGAAGAATATATGGATTATACCTGAGCGTAACGTTATTGGTACGACACCGCAAGATTTTCATCCACCTACTTCGTTGAAGAACGGTGACTCTTCCTATTACGACCCCAATTATCTCCAGTCGGATGAAGAGAAGGACAGATTCCTTAAAATAGTAACCAAAATCTTTAACAGGATTAATAACAATCTAGCCGGAAGTATTTTGCTTGAAGAGCTTAGTAAAGCTAATCCTTACCTAGGTAACGATGATACACCAGACAACCAGTTTCATATAGGCGATGCATCCGCCGTGGAAATCAAATTTAGCAATGGATCACAGCATATTCTCTTGCCCAACGTTATTATAATGGGGGCGGAACCAAATTTATTTTTGACATATCAGAGAAATATTAGCCTGAGAAATAACTATAAGCCGTCAAACCATGGGTTCGGTAGCATAGCAATCGTTACTTTTTCTCCCGAATACAGTTTTCGCTTCAATGATAATAGTATAAATGAGTTTATCCAAGACCCCGCACTCACGCTTATGCACGAACTCATACACTCTTTACACGGCCTGTATGGCGCTAAGGGGATAACCACTACGTGTACCATTACTCAGCAACAGAACCCATTGATAACGAACAGGAAGGGCATTAAAATCGAGGAATTTCTTACATTTGGAGGCAACGATCTGAACATTATAACTGTCGCACAGTACAATGACATCTATACCAACTTACTAAATGATTATAGAAAAATCGCTTCTAAGTTATCCAAGGTTCAAGTCTCAAACCCTCAACTGAATCCGTATAAGGACATATTCCAAGAAAAATATGGATTAGACAAAGACGCGTCAGGAATCTATTCGGTAAACATTAACAAATTCGACGATATTTTGAAGAAACTTTACAGCTTCACGGAGTTCGACTTGGCCACCAAATTCCAGGTCAAATGCCGAGAGACATACATCGGACGGCCTAAGTTTTTTAAGCTGTCGAATCTCCTGAATGATTCCATATACAACATTAGTGAGGGTTACAATATAAATAACCTAAAGGTGAATTTCCGAGGCCAAAACGCCAACCTAAATCCGCGTATCATTAAACCCATCACAGGACGGGGGTTAGTGAAGAAAATAATCCGGTTT

SEQ ID NO: 9 - protein, 1233 aa, Artificial Sequence
  Synthetic
ATGCCAAAAATCAACAGCTTTAATTACAATGACCCTGTAAACGATCGTACCATCCTATACATAAAGCCGGGTGGGTATCACGAGTTCTACAAATCTTTCAATATTATGAAGAATATATGGATTATACCTGAGCGTAACGTTATTGGTACGACACCGCAAGATTTTCATCCACCTACTTCGTTGAAGAACGGTGACTCTTCCTATTACGACCCCAATTATCTCCAGTCGGATGAAGAGAAGGACAGATTCCTTAAAATAGTAACCAAAATCTTTAACAGGATTAATAACAATCTAGCCGGAAGTATTTTGCTTGAAGAGCTTAGTAAAGCTAATCCTTACCTAGGTAACGATGATACACCAGACAACCAGTTTCATATAGGCGATGCATCCGCCGTGGAAATCAAATTTAGCAATGGATCACAGCATATTCTCTTGCCCAACGTTATTATAATGGGGGCGGAACCAAATTTATTTTTGACATATCAGAGAAATATTAGCGCCAGAAATAACTATAAGCCGTCAAACCATGGGTTCGGTAGCATAGCAATCGTTACTTTTTCTCCCGAATACAGTTTTCGCTTCAATGATAATAGTATAAATGAGTTTATCCAAGACCCCGCACTCACGCTTATGCACGAACTCATACACTCTTTACACGGCCTGTATGGCGCTAAGGGGATAACCACTACGTGTACCATTACTCAGCAACAGAACCCATTGATAACGAACAGGAAGGGCATTAAAATCGAGGAATTTCTTACATTTGGAGGCAACGATCTGAACATTATAACTGTCGCACAGTACAATGACATCTATACCAACTTACTAAATGATTATAGAAAAATCGCTTCTAAGTTATCCAAGGTTCAAGTCTCAAACCCTCAACTGAATCCGTATAAGGACATATTCCAAGAAAAATATGGATTAGACAAAGACGCGTCAGGAATCTATTCGGTAAACATTAACAAATTCGACGATATTTTGAAGAAACTTTACAGCTTCACGGAGTTCGACTTGGCCACCAAATTCCAGGTCAAATGCCGAGAGACATACATCGGACGGCCTAAGTTTTTTAAGCTGTCGAATCTCCTGAATGATTCCATATACAACATTAGTGAGGGTTACAATATAAATAACCTAAAGGTGAATTTCCGAGGCCAAAACGCCAACCTAAATCCGCGTATCATTAAACCCATCACAGGACGGGGGTTAGTGAAGAAAATAATCCGGTTT

SEQ ID NO: 10 - protein, 1758 aa, Artificial Sequence
  Synthetic
ATGGACTCGGGCCGGGACTTCCTGACCCTGCACGGCCTACAGGATGATGAGGATCTACAGGCGCTGCTGAAGGGCAGCCAGCTCCTGAAGGTGAAGTCCAGCTCATGGAGGAGAGAGCGCTTCTACAAGTTGCAGGAGGACTGCAAGACCATCTGGCAGGAGTCCCGCAAGGTCATGCGGACCCCGGAGTCCCAGCTGTTCTCCATCGAGGACATTCAGGAGGTGCGAATGGGGCACCGCACGGAGGGTCTGGAGAAGTTCGCCCGTGATGTGCCCGAGGACCGCTGCTTCTCCATTGTCTTCAAGGACCAGCGCAATACACTAGACCTCATCGCCCCATCGCCAGCTGATGCCCAGCACTGGGTGCTGGGGCTGCACAAGATCATCCACCACTCAGGCTCCATGGACCAGCGTCAGAAGCTACAGCACTGGATTCACTCCTGCTTGCGAAAAGCTGACAAAAACAAGGACAACAAGATGAGCTTCAAGGAGCTGCAGAACTTCCTGAAGGGTGGTGGTGGTAGCATGCCAAAAATCAACAGCTTTAATTACAATGACCCTGTAAACGATCGTACCATCCTATACATAAAGCCGGGTGGGTATCACGAGTTCTACAAATCTTTCAATATTATGAAGAATATATGGATTATACCTGAGCGTAACGTTATTGGTACGACACCGCAAGATTTTCATCCACCTACTTCGTTGAAGAACGGTGACTCTTCCTATTACGACCCCAATTATCTCCAGTCGGATGAAGAGAAGGACAGATTCCTTAAAATAGTAACCAAAATCTTTAACAGGATTAATAACAATCTAGCCGGAAGTATTTTGCTTGAAGAGCTTAGTAAAGCTAATCCTTACCTAGGTAACGATGATACACCAGACAACCAGTTTCATATAGGCGATGCATCCGCCGTGGAAATCAAATTTAGCAATGGATCACAGCATATTCTCTTGCCCAACGTTATTATAATGGGGGCGGAACCAAATTTATTTTTGACATATCAGAGAAATATTAGCCTGAGAAATAACTATAAGCCGTCAAACCATGGGTTCGGTAGCATAGCAATCGTTACTTTTTCTCCCGAATACAGTTTTCGCTTCAATGATAATAGTATAAATGAGTTTATCCAAGACCCCGCACTCACGCTTATGCACGAACTCATACACTCTTTACACGGCCTGTATGGCGCTAAGGGGATAACCACTACGTGTACCATTACTCAGCAACAGAACCCATTGATAACGAACAGGAAGGGCATTAAAATCGAGGAATTTCTTACATTTGGAGGCAACGATCTGAACATTATAACTGTCGCACAGTACAATGACATCTATACCAACTTACTAAATGATTATAGAAAAATCGCTTCTAAGTTATCCAAGGTTCAAGTCTCAAACCCTCAACTGAATCCGTATAAGGACATATTCCAAGAAAAATATGGATTAGACAAAGACGCGTCAGGAATCTATTCGGTAAACATTAACAAATTCGACGATATTTTGAAGAAACTTTACAGCTTCACGGAGTTCGACTTGGCCACCAAATTCCAGGTCAAATGCCGAGAGACATACATCGGACGGCCTAAGTTTTTTAAGCTGTCGAATCTCCTGAATGATTCCATATACAACATTAGTGAGGGTTACAATATAAATAACCTAAAGGTGAATTTCCGAGGCCAAAACGCCAACCTAAATCCGCGTATCATTAAACCCATCACAGGACGGGGGTTAGTGAAGAAAATAATCCGGTTT

SEQ ID NO: 11 - protein, 1758 aa, Artificial Sequence
  Synthetic
ATGGACTCGGGCCGGGACTTCCTGACCCTGCACGGCCTACAGGATGATGAGGATCTACAGGCGCTGCTGAAGGGCAGCCAGCTCCTGAAGGTGAAGTCCAGCTCATGGAGGAGAGAGCGCTTCTACAAGTTGCAGGAGGACTGCAAGACCATCTGGCAGGAGTCCCGCAAGGTCATGCGGACCCCGGAGTCCCAGCTGTTCTCCATCGAGGACATTCAGGAGGTGCGAATGGGGCACCGCACGGAGGGTCTGGAGAAGTTCGCCCGTGATGTGCCCGAGGACCGCTGCTTCTCCATTGTCTTCAAGGACCAGCGCAATACACTAGACCTCATCGCCCCATCGCCAGCTGATGCCCAGCACTGGGTGCTGGGGCTGCACAAGATCATCCACCACTCAGGCTCCATGGACCAGCGTCAGAAGCTACAGCACTGGATTCACTCCTGCTTGCGAAAAGCTGACAAAAACAAGGACAACAAGATGAGCTTCAAGGAGCTGCAGAACTTCCTGAAGGGTGGTGGTGGTAGCATGCCAAAAATCAACAGCTTTAATTACAATGACCCTGTAAACGATCGTACCATCCTATACATAAAGCCGGGTGGGTATCACGAGTTCTACAAATCTTTCAATATTATGAAGAATATATGGATTATACCTGAGCGTAACGTTATTGGTACGACACCGCAAGATTTTCATCCACCTACTTCGTTGAAGAACGGTGACTCTTCCTATTACGACCCCAATTATCTCCAGTCGGATGAAGAGAAGGACAGATTCCTTAAAATAGTAACCAAAATCTTTAACAGGATTAATAACAATCTAGCCGGAAGTATTTTGCTTGAAGAGCTTAGTAAAGCTAATCCTTACCTAGGTAACGATGATACACCAGACAACCAGTTTCATATAGGCGATGCATCCGCCGTGGAAATCAAATTTAGCAATGGATCACAGCATATTCTCTTGCCCAACGTTATTATAATGGGGGCGGAACCAAATTTATTTTTGACATATCAGAGAAATATTAGCGCCAGAAATAACTATAAGCCGTCAAACCATGGGTTCGGTAGCATAGCAATCGTTACTTTTTCTCCCGAATACAGTTTTCGCTTCAATGATAATAGTATAAATGAGTTTATCCAAGACCCCGCACTCACGCTTATGCACGAACTCATACACTCTTTACACGGCCTGTATGGCGCTAAGGGGATAACCACTACGTGTACCATTACTCAGCAACAGAACCCATTGATAACGAACAGGAAGGGCATTAAAATCGAGGAATTTCTTACATTTGGAGGCAACGATCTGAACATTATAACTGTCGCACAGTACAATGACATCTATACCAACTTACTAAATGATTATAGAAAAATCGCTTCTAAGTTATCCAAGGTTCAAGTCTCAAACCCTCAACTGAATCCGTATAAGGACATATTCCAAGAAAAATATGGATTAGACAAAGACGCGTCAGGAATCTATTCGGTAAACATTAACAAATTCGACGATATTTTGAAGAAACTTTACAGCTTCACGGAGTTCGACTTGGCCACCAAATTCCAGGTCAAATGCCGAGAGACATACATCGGACGGCCTAAGTTTTTTAAGCTGTCGAATCTCCTGAATGATTCCATATACAACATTAGTGAGGGTTACAATATAAATAACCTAAAGGTGAATTTCCGAGGCCAAAACGCCAACCTAAATCCGCGTATCATTAAACCCATCACAGGACGGGGGTTAGTGAAGAAAATAATCCGGTTT

SEQ ID NO: 12 - protein, 403 aa, Homo sapiens [sequence omitted]

SEQ ID NO: 13 - protein, 20 aa, Artificial Sequence
  Synthetic
NGSLCDQEIDSICSIMEKAD

SEQ ID NO: 14 - protein, 438 aa, Artificial Sequence
  Synthetic
CKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKS

SEQ ID NO: 15 - protein, 403 aa, Artificial Sequence
  Synthetic
SSVLNMRYKNDKYVDTSGYDSNININGDVYKYPTNKNQFEIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 16 - protein, 206 aa, Artificial Sequence
  Synthetic
MAEDADMRNELEEMQRRADQLADESLESTRRMLQLVEESKDAGIRTLVMLDEQGEQLERIEEGMDQINKDMKEAEKNLTDLGKFCGLCVCPCNKLKSSDAYKKAWGNNQDGVVASQPARVVDEREQMAISGGFIRRVTNDARENEMDENLEQVSGIIGNLRHMALDMGNEIDTQNRQIDRIMEKADSNKTRIDEANQRATKMLGSG

SEQ ID NO: 17 - protein, 10 aa, Artificial Sequence
  Synthetic
RQIDRIMEKA

SEQ ID NO: 18 - protein, 118 aa, Homo sapiens
NPDREGWLLKLGGGRVKTWKRRWFILTDNCLYYFEYTTDKEPRGIIPLENLSIREVEDSKKPNCFELYIPDNKDQVIKACKTEADGRVVEGNHTVYRISAPTPEEKEEWIKCIKAAIS

SEQ ID NO: 19 - protein, 118 aa, Homo sapiens
NPDREGWLLKLGGGRVKTWKRRWFILTDNCLYYFEYTTDKEPRGIIPLENLSIREVDDPRKPNCFELYIPNNKGQLIKACKTEADGRVVEGNHMVYRISAPTQEEKDEWIKSIQAAVS

SEQ ID NO: 20 - protein, 118 aa, Homo sapiens
NPDREGWLLKLGGGRVKTWKRRWFILTDNCLYYFEYTTDKEPRGIIPLENLSIREVEDPRKPNCFELYNPSHKGQVIKACKTEADGRVVEGNHVVYRISAPSPEEKEEWMKSIKASIS

SEQ ID NO: 21 - protein, 131 aa, Homo sapiens
AVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIR

SEQ ID NO: 22 - DNA, 1209 bp, Homo sapiens
atgacagccatcatcaaagagatcgttagcagaaacaaaaggagatatcaagaggatggattcgacttagacttgacctatatttatccaaacattattgctatgggatttcctgcagaaagacttgaaggcgtatacaggaacaatattgatgatgtagtaaggtttttggattcaaagcataaaaaccattacaagatatacaatctttgtgctgaaagacattatgacaccgccaaatttaattgcagagttgcacaatatccttttgaagaccataacccaccacagctagaacttatcaaacccttttgtgaagatcttgaccaatggctaagtgaagatgacaatcatgttgcagcaattcactgtaaagctggaaagggacgaactggtgtaatgatatgtgcatatttattacatcggggcaaatttttaaaggcacaagaggccctagatttctatggggaagtaaggaccagagacaaaaagggagtaactattcccagtcagaggcgctatgtgtattattatagctacctgttaaagaatcatctggattatagaccagtggcactgttgtttcacaagatgatgtttgaaactattccaatgttcagtggcggaacttgcaatcctcagtttgtggtctgccagctaaaggtgaagatatattcctccaattcaggacccacacgacgggaagacaagttcatgtactttgagttccctcagccgttacctgtgtgtggtgatatcaaagtagagttcttccacaaacagaacaagatgctaaaaaaggacaaaatgtttcacttttgggtaaatacattcttcataccaggaccagaggaaacctcagaaaaagtagaaaatggaagtctatgtgatcaagaaatcgatagcatttgcagtatagagcgtgcagataatgacaaggaatatctagtacttactttaacaaaaaatgatcttgacaaagcaaataaagacaaagccaaccgatacttttctccaaattttaaggtgaagctgtacttcacaaaaacagtagaggagccgtcaaatccagaggctagcagttcaacttctgtaacaccagatgttagtgacaatgaacctgatcattatagatattctgacaccactgactctgatccagagaatgaaccttttgatgaagatcagcatacacaaattacaaaagtc

SEQ ID NO: 23 - DNA, 354 bp, Homo sapiens
aatccagaccgagaaggctggctattgaaactcggaggtggcagggtaaagacttggaagagacgctggttcattctgactgacaactgcctttactactttgagtataccacggataaggagccccgtggaatcatccctttagagaatctgagtatccgggaagtggaggactccaaaaaaccaaactgctttgagctttatatccccgacaataaagaccaagttatcaaggcctgcaagaccgaggctgacgggcgggtggtggaggggaaccacactgtttaccggatctcagctccgacgcccgaggagaaggaggagtggattaagtgcattaaagcagccatcagc

SEQ ID NO: 24 - DNA, 354 bp, Homo sapiens
aacccggaccgggagggctggctcctgaagctggggggcgggcgggtgaagacgtggaagcggcgctggtttatcctcacagacaactgcctctactactttgagtacaccacggacaaggagccccgaggaatcatccccctggagaatctgagcatccgagaggtggacgacccccggaaaccgaactgctttgaactttacatccccaacaacaaggggcagctcatcaaagcctgcaaaactgaggcggacggccgagtggtggagggaaaccacatggtgtaccggatctcggcccccacgcaggaggagaaggacgagtggatcaagtccatccaggcggctgtgagt

SEQ ID NO: 25 - DNA, 354 bp, Homo sapiens
aaccccgaccgcgagggctggctcctgaagctgggagggggccgtgtgaagacctggaagcgccggtggttcatcctgaccgataactgcctctattactttgaatacacaacagataaggagcccaggggaatcatcccgttggaaaacctcagcatcagggaggtggaggacccccggaaacccaactgttttgagctctacaatcccagccacaaagggcaggtcatcaaggcctgtaagactgaggccgacggccgcgtggtagaggggaaccatgtggtgtaccggatctcagccccgagcccggaggagaaggaggagtggatgaaatccatcaaagccagtatcagc

SEQ ID NO: 26 - DNA, 393 bp, Homo sapiens
gcagtgattctggagagcatctttctgaagcgatcccaacagaaaaagaaaacatcacctctaaacttcaagaagcgcctgtttctcttgaccgtgcacaaactctcctactatgagtatgactttgaacgtgggagaagaggcagtaagaagggttcaatagatgttgagaagatcacttgtgttgaaacagtggttcctgaaaaaaatcctcctccagaaagacagattccgagaagaggtgaagagtccagtgaaatggagcaaatttcaatcattgaaaggttcccttatcccttccaggttgtatatgatgaagggcctctctacgtcttctccccaactgaagaactaaggaagcggtggattcaccagctcaaaaacgtaatccgg

SEQ ID NO: 27 - protein, 389 aa, Artificial Sequence
  Synthetic
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSGGSGGSENGSLCDQEIDS

SEQ ID NO: 28 - protein, 228 aa, Artificial Sequence
  Synthetic
ICSIERADNGGSGGSPPYTITYFPVRGRCEAMRMLLADQDQSWKEEVVTMETWPPLKPSCLFRQLPKFQDGDLTLYQSNAILRHLGRSFGLYGKDQKEAALVDMVNDGVEDLRCKYATLIYTNYEAGKEKYVKELPEHLKPFETLLSQNQGGQAFVVGSQISFADYNLLDLLRIHQVLNPSCLDAFPLLSAYVARLSARPKIKAFLASPEHVNRPINGNGKGHHHHHH